(19) United States
(12) Patent Application Publication
(10) Pub. No.: US 2024/0299771 A1
(43) Pub. Date: Sep. 12, 2024

(54) NON-INVASIVE, DYNAMIC, MULTI-WAVELENGTH, MULTI-NODE PHOTOBIOMODULATION THERAPY METHODS AND SYSTEMS FOR TREATMENT OF COMPLEX DISEASES

(71) Applicant: Polytone Laser Inc., Montreal (CA)

(72) Inventors: Luis Alberto Santana Blank, Montreal (CA); Elizabeth Rodriguez De Santana, Montreal (CA)

(21) Appl. No.: 18/591,750

(22) Filed: Feb. 29, 2024

Related U.S. Application Data
(60) Provisional application No. 63/450,215, filed on Mar. 6, 2023.

(51) Int. Cl. A61N 5/06

(52) U.S. Cl. CPC A61N 5/062; A61N 2005/0629; A61N 2005/063; A61N 2005/0642; A61N 2005/0659; A61N 2005/0663

(57) ABSTRACT

Noninvasive, dynamic, multi-wavelength, multi-pulse, multi-node photobiomodulation methods can be used to treat complex diseases (CDs), including, but not limited to cancer, diseases of the eye or brain, and arterial disease. Light is non-invasively provided to the skin of a body proximal a plurality of arteries. In various implementations, the optical signals applied to the body and/or the parameters of the regimen for applying the optical signals to the body are fractal in nature and/or are similar to or match fractals or characteristics of fractals in one or more biological signals, system, structures or processes.

### CROSS-REFERENCE TO RELATED PATENT APPLICATIONS

This application claims the benefit of priority of U.S. Provisional Application No. 63/450,215 titled “NON-INVASIVE, DYNAMIC, MULTI-WAVELENGTH, MULTI-NODE PHOTOBIOMODULATION THERAPY METHODS AND SYSTEMS FOR TREATMENT OF COMPLEX DISEASES,” filed Mar. 6, 2023. The entirety of each application referenced in this paragraph is incorporated herein by reference.

### BACKGROUND OF THE INVENTION

### Field

The present disclosure relates to use of non-ionizing electromagnetic (EM), e.g., optical, signals for medical treatment, and more specifically, to photobiomodulation (PBM) therapy such as for treatment of complex diseases (CD).

### Description of the Related Art

From the pharmaceutical perspective, to avoid non-specific toxicity, the non-surgical treatment of CDs generally relies on agents targeting single molecular targets. However, diseased tissues can often adapt to variations in one molecular pathway, leading to partial or transitory responses followed by progression or relapse. The former is often observed in the treatment of peripheral arterial disease (PAD) and CDs of the eye and brain, whereas the latter is particularly found in intermediate and late-stage solid tumors.

What are needed therefore are alternative methods of treating CD such a diseases of the eye and brain as well as effective tools for addressing intermediate and late-stage tumors.

### SUMMARY

Various innovations disclosed herein comprise noninvasive, dynamic, multi-wavelength, multi-pulse, multi-node PBM methods and systems for the treatment of complex diseases (CDs), including, but not limited to cancer (i.e., solid tumors, not including myeloid diseases), diseases of the eye (e.g., glaucoma, age-related macular degeneration (AMD), diabetic retinopathy (DR), retinitis pigmentosa (RP)), brain (e.g., Alzheimer's (AD) and Parkinson's disease (PD)), and arterial diseases (e.g., peripheral arterial disease (PAD)).

The present disclosure relates generally to methods for non-invasively providing light to the skin of a body to provide medical treatment. In various implementations, the light is provided to the skin of a body proximal one or more of arteries.

The mathematical, physical, chemical, biological, and computational bases of the present disclosure support an original method for absorption, transport, and transduction of radiant (photic) energy, locally and at a distance, at nano to macro scales, aiming to power, couple with and synchronize physiologic rhythms (PRs) to reestablish homeostasis/homeokinesis (i.e., healthy state).

PRs exist in myriad spatio-temporal scales within tissues, which are energy-dependent constructs made up of biomolecules and water. Pulsed energy signals such as those described in the present disclosure may help power and reestablish altered PRs as shown by the fact that oscillatory states can carry signals for temporal coding, and pulsating signals have transduction functions in biological systems.

Moreover, the human body is a non-equilibrium, non-linear, dissipative system wherein fractals are structurally and functionally ingrained as extensively documented in the scientific literature. Consequently, the fractal characteristics described in the present disclosure may have deep repercussions in the treatment of the referred CDs.

For example, various methods of treating a human or animal having a body using pulses of light disclosed herein may comprise non-invasively applying a first sequence of optical pulses comprising at least a first plurality of pulses of a first wavelength at a first repetition rate, and subsequently, a second plurality of pulses of a second different wavelength at a second repetition rate to a plurality of anatomical locations on the body as part of the first sequence for a first session on a first day. (The term “first” in this context does not preclude application of other prior pulses in the same sequence or previous sequences, in the same session or previous sessions on the same day or on previous days nor inclusion of other prior sequences, sessions or days, but is used as a matter of convenience for identification of an action, step, item, element, component or group, etc.) The plurality of anatomical locations is exterior of the body and proximal to arteries. In various implementations, the first and second repetition rates are in a range from 1 to 15 MHz.

Various methods described herein also include a method of treating a human or animal having a body using pulses of light wherein the method comprises non-invasively applying modulated light to a plurality of anatomical locations on the body simultaneously wherein the plurality of anatomical locations includes at least two of the following:
(a) a location on the skin proximal the left carotid artery and a location on the skin proximal the right carotid artery,
(b) a location on the skin proximal the left axillary artery and a location on the skin proximal the right axillary artery,
(c) a location on the skin proximal the left femoral artery and a location on the skin proximal the right femoral artery,
(d) a location on the skin proximal the left popliteal artery and a location on the skin proximal the right popliteal artery,
(e) a location on the skin proximal the left tibial artery and a location on the skin proximal the right tibial artery,
(f) a location on the skin proximal the left dorsalis pedis artery and a location on the skin proximal the right dorsalis pedis artery,
(g) a location on the skin proximal the left radial artery and a location on the skin proximal the right radial artery,
(i) a location on the skin proximal the left iliac artery and a location on the skin proximal the right iliac artery, or
(j) a location on the skin proximal the left posterior tibial artery and a location on the skin proximal the right posterior tibial artery.

Some methods described herein include a method of treating a human or animal having a body using pulses of light wherein the method comprises non-invasively applying a plurality of sequences of optical pulses comprising at least a first plurality of pulses of a first (e.g., central) wavelength at a first repetition rate to a plurality of anatomical locations on the body as part of a plurality of sessions on one or more days, wherein parameters of the plurality of sequences of optical pulses are fractals and/or exhibit properties of fractals. (As discussed above, the term “first” in this context does not preclude application of pulses prior to application of the optical pulses of a first wavelength and a first repetition rate in the same sequence or previous sequences, in the same session or previous sessions on the same day or on previous days, nor inclusion of other prior sequences, sessions or days, but is used as a matter of convenience for identification of an action, step, item, element, component or group, etc.)

Some methods described herein include a method of treating a human or animal having a body using pulses of light wherein the method comprises non-invasively applying a first sequence of optical pulses comprising at least a first plurality of pulses of a first (e.g., central) wavelength at a first repetition rate, and subsequently a second plurality of pulses of a second different (e.g., central) wavelength at a second repetition rate to a plurality of anatomical locations on the body as part of the first sequence for a first session on a first day, and wherein the first and second repetition rates are in a range from 1 to 15 MHz. (As discussed above, the term “first” in this context does not preclude application of pulses prior to application of the optical pulses of a first wavelength and a first repetition rate in the same sequence or previous sequences, in the same session or previous sessions on the same day or on previous days, nor inclusion of other prior sequences, sessions or days, but is used as a matter of convenience for identification of an action, step, item, element, component or group, etc.)

A wide variety of variations and other methods, however, may be employed.

Quantities or values recited herein are meant to refer to the actual given value. The term “about” is used herein to refer to the approximation to such given value that would reasonably be inferred based on the ordinary skill in the art, including equivalents and approximations due to the experimental and/or measurement conditions for such given value. The functions of the various elements shown in the figures, including any functional block labeled as a “controller” and/or “electronics” (or control electronics), may be provided through the use of dedicated hardware as well as hardware capable of executing software in association with appropriate software. When operation of the controller and/or electronics (or control electronics) is provided by a processor, the functions may be provided by a single dedicated processor, by a single shared processor, or by a plurality of individual processors, some of which may be shared. In some embodiments of the present technology, the processor may be a general-purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

The examples and conditional language recited herein are principally intended to aid the reader in understanding the principles of the present technology and not to limit its scope to such specifically recited examples and conditions. It will be appreciated that those skilled in the art may devise various systems that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Embodiments of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 is a perspective view of a plurality of light sources configured to non-invasively provide light to a plurality of locations proximal arteries of a human body such as locations on the skin proximal left and right carotid arteries, left and right axillary arteries, left and right femoral arteries, left and right popliteal arteries, left and right anterior tibial arteries, and left and right dorsalis pedis arteries.

FIG. 2 is a perspective view of a plurality of light sources configured to non-invasively provide light to a plurality of locations proximal arteries of a human body such as locations on the skin proximal left and right radial arteries, left and right iliac arteries, left and right posterior tibial arteries.

FIG. 3 is a block diagram illustrating an example method for producing and/or executing a regimen of light treatment or therapy.

FIG. 4A is a block diagram of a method of exposing a subject to light wherein, for example, the parameters of the regimen are selected or established such that, the variation of the parameters in the regimen comprises one or more fractals or exhibits one or more characteristics of one or more fractals.

FIG. 4B is a block diagram of a method of exposing a subject to light wherein, for example, the parameters of the regimen are selected or established such that the variation of the parameters in the regimen comprises one or more fractals or exhibits one or more characteristics of one or more fractals within a threshold difference of one or more fractals or characteristics of one or more fractals for a biological signal, system, structure or process of the subject.

It should also be noted that, unless otherwise explicitly specified herein, the drawings are not drawn to scale and are not anatomically and/or topographically exact.

### DETAILED DESCRIPTION OF CERTAIN INVENTIVE ASPECTS

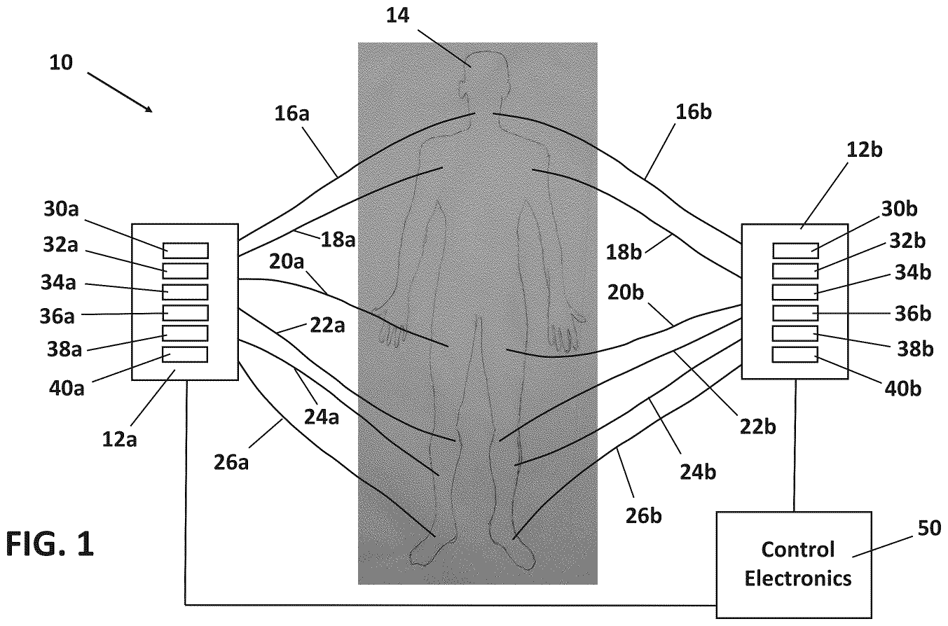

Various methods of providing medical treatment and/or therapy described herein include application of light such as optical pulses. Such light may comprise laser light in some implementations. Such light may be modulated and be in the form of pulses in various cases. FIG. 1 schematically illustrates an example system 10 configured to implement such a method, for example, on a human body 14. While the subject as generally described herein is a human subject, it is contemplated that animals, including other mammals, could be a light-receiving subject. In the example shown, the system 10 is configured to deliver a non-invasive (e.g., applied to the skin), non-thermal (e.g., temperature increases <0.01° C.), and non-ionizing light treatment to the body 14. In this example, the light is applied to the skin proximal arteries such as arteries of the body. In various implementations, for example, the light is delivered to the skin proximal one or more of the following arteries: the left carotid artery, the right carotid artery, the left axillary artery, the right axillary artery, the left femoral artery, the right femoral artery, the left popliteal artery, the right popliteal artery, the left anterior tibial artery, the right anterior tibial artery, the left dorsalis pedis artery, the right dorsalis pedis artery or any combination. In various implementations, light such as optical pulses are delivered to locations proximal to both the left and the right arteries, respectively, such as locations proximal both the left and right carotid arteries, left and right axillary arteries, left and right femoral arteries, left and right popliteal arteries, left and right anterior tibial arteries, left and right dorsalis pedis arteries or any combination thereof. Application to the same anatomical feature (e.g., the same artery) on both the left and right side of the body and/or the same location on the left and right side of the body may be referred to herein as symmetrical such as symmetrical application of light (e.g., optical pulses) to the body. Delivery of light to a plurality of locations may be referred to herein as multi-nodal or multi-node. Node selection is based on the principles of non-lineal (chaotic) transport. As mathematically described by Meiss, transport velocity can be increased fourfold between sites in an orbital (epsilon) segment wherein each node acts as a repeater, in accordance with the principles of quantic symmetry, fractality and transport in chaotic systems.

Accordingly, various methods and systems 10 disclosed herein such as schematically illustrated with FIG. 1 include non-invasive, dynamic, multi-wavelength, multi-node photobiomodulation (PBM) methods and systems that dynamically emit light such as laser light (e.g., diode laser light) signals with different (e.g., central) wavelengths, ranging in some cases, from visible light (e.g., yellow, and red) to infrared (i.e., NIR, MIR, and FIR). Dynamically may refer, inter alia, to the ability to alter which wavelength of light is applied. As referenced above, this light can be modulated to produce pulses. This modulation may be high-frequency such as possibly in the range of 1-15 MHz or 1-10 MHz (e.g., pulses in the high frequency RF range and/or in the ultrasonic frequency range). In some implementations, the modulation frequency may be different for different wavelength signals or different wavelengths of light. Switching between light having different modulation frequencies may also be referred to as dynamic. In various methods and designs, energy is simultaneously delivered by one or more nodes or terminals, for example, comprising optic fibers or optical fiber bundles 16a, 16b, 18a, 18b, 20a, 20b, 22a, 22b, 24a, 24b, 26a, 26b and/or other delivery systems to various locations of the body. As discussed above, terminals may, for example, be applied symmetrically non-invasively to the skin on or above or proximal arteries.

As referenced above, in some implementations, the sites of the output terminals (e.g., distal ends of the optical fibers or optical fiber bundles) and/or the locations where light is directed may be based on the vascular system, and the Biologically Closed Electric Circuit (BCEC) and its sub-set Vascular Interstitial Closed Circuit (VICC) that are conceived to exist with the wall of arteries and veins functioning as relative insulators around the electrically conducting medium of blood, the plasma. Moreover, in some implementations, the sites of the output terminals (e.g., distal ends of the optical fibers or fiber bundles) and/or target areas where light is applied may be based on the fractal geometry of vascular system, and/or the Biologically Closed Electric Circuit (BCEC) and/or its sub-set Vascular Interstitial Closed Circuit (VICC), and/or intramural submucosae of blood vessels and pre-lymphatic reticular anatomic structures described by Thiese et al. The latter intramural circulation system transports fluids, biomolecules, cells and produces stratification of water and separation of charges in the collagen hemi-wall, creating a bio-battery, which may release electrical charges with implications—for instance—in the destruction of tumor cells.

Without subscribing to any particular scientific theory, in various cases the method uses photic energy (light or photons) that may travel through the plasma of the blood exerting a non-thermal photochemical and photophysical effect while interacting with the electrons of water ions (H⁺, OH⁻). Additionally, there is an annular layer of structured water covering the interior of all vessels, that adheres to their hydrophilic (electronegative) surface. The layer of structured water has charge separation and thus, it is an energy reservoir that can behave as a biobattery or exclusion zone (EZ). Again, without subscribing to any particular theory, the application of light in the BCEC-VICC can transfer radiant energy, which recharges the reservoir of potential energy represented by the biobattery. As a result, this method may allow the transport of energy at distance. The reservoir remains stable, unless it is disturbed by injury redox potentials. In that scenario, it may potentially release, for example, up to 70% of its electric charge.

As illustrated in FIG. 1, the system 10 may comprise first and second laser assemblies 12a, 12b each comprising a plurality of light sources 30a, 30b, 32a, 32b, 34a, 34b, 36a, 36b, 38a, 38b, 40a, 40b. The light sources 30a, 30b, 32a, 32b, 34a, 34b, 36a, 36b, 38a, 38b, 40a, 40b may comprise lasers. For example, the light sources 30a, 30b, 32a, 32b, 34a, 34b, 36a, 36b, 38a, 38b, 40a, 40b may comprise laser diodes.

As illustrated, the first laser assembly 12a includes a first plurality of light sources 30a, 32a, 34a, 36a, 38a, 40a and the second laser assembly 12b includes a second plurality of light sources 30b, 32b, 34b, 36b, 38b, 40b.

A plurality of optical fibers or optical fiber bundles 16a, 16b, 18a, 18b, 20a, 20b, 22a, 22b, 24a, 24b, 26a, 26b are shown optically coupled from the first and second laser assemblies 12a, 12b. In particular, a first group of optical fiber or optical fiber bundles 16a, 18a, 20a, 22a, 24a, 26a is optically coupled to the first laser assembly 12a, and a second group of optical fibers or optical fiber bundles 16b, 18b, 20b, 22b, 24b, 26b is optically coupled to the second laser assembly 12b. The optical fibers or optical fiber bundles 16a, 16b, 18a, 18b, 20a, 20b, 22a, 22b, 24a, 24b, 26a, 26b have proximal and distal ends. The proximal end is closer to the laser assembly 12a, 12b than the distal end. Moreover, the proximal end is shown coupled to the laser assembly 12a, 12b. The distal ends are shown closer to the body 14 than the proximal ends. The distal ends may form a terminal or node or part thereof or feed into a terminal or node. In some designs, for example, the terminal or node where the light is output from the distal end of the optical fiber(s)/optical fiber bundle, includes one or more components to facilitate delivery of light to the body. In some designs, for example, a lens or possible other optical component or components may be included at the distal end or at the node or terminal. In some embodiments, at least one adhesive pad is connected to the distal end of the at least one fiber bundle; and when the at least one adhesive pad is applied to the subject, the at least one adhesive pad is configured to position the distal end of the at least one fiber bundle such that light from the plurality of light sources (e.g., laser diodes) is delivered to the subject when the device is in use. Additionally or alternatively to adhesive pads, connection or application can include, but are not limited to use of: cuffs, tape, straps, belts, bracelets, and necklaces. In some implementations, a beam is directed to the target areas wherein the beam has a 5-10 mm cross-sectional width or diameter. Likewise, in various implementations, the area of the body at the target location, e.g., at the node or terminal such as at the distal end of the fiber bundles or fibers (e.g., proximal to arteries or skin above the arteries) that is exposed to the light is between 5-10 mm. The area of the body at the target location, e.g., at the node or terminal such as at the distal end of the fiber bundles or fibers (e.g., proximal to arteries or skin above the arteries) that is exposed to the light from the light source is in various implementations less than 100 cm², 50 cm², 40 cm², 20 cm², 10 cm², 5 cm², 4 cm², 3 cm², 2 cm², 1 cm², 50 mm², 25 mm², 20 mm², 10 mm², 5 mm², or in any range formed by any of these values, for example, from 10 mm² to 2 cm² or from 20 mm² to 3 cm², or is possibly larger or smaller. In some implementations, the light from the light sources can be applied to the target locations, regions, or sites, e.g., at the node or terminal such as at the distal end of the fiber bundles or fibers, (e.g., proximal to arteries or skin above the arteries) and not elsewhere on the body.

As illustrated, the first plurality of optical fibers or optical fiber bundles 16a, 18a, 20a, 22a, 24a, 26a are directed to a first side of the body, e.g., the right side of the body (from the subject's viewpoint). Similarly, the second plurality of optical fibers or optical fiber bundles 16b, 18b, 20b, 22b, 24b, 26b are directed to a second side of the body, e.g., the left side of the body (from the subject's viewpoint). Likewise, in this particular design, the first laser assembly 12a is optically coupled to optical fibers and/or optical fiber bundles 16a, 18a, 20a, 22a, 24a, 26a configured to direct light to the first side (e.g., right side) of the body 14 and the second laser assembly 12b is optically coupled to optical fibers and/or optical fiber bundles 16b, 18b, 20b, 22b, 24b, 26b configured to direct light to the second side of the body (e.g., the left side from the perspective of the subject). Other configurations are possible. For example, a single laser assembly to which the optical fibers or optical fiber bundles 16a, 16b, 18a, 18b, 20a, 20b, 22a, 22b, 24a, 24b, 26a, 26b are coupled may be employed. Alternatively, more laser assemblies may be employed. Additionally, which optical fibers or fiber bundles are optically coupled to which laser assembly 12a, 12b may be different. Likewise, the distribution of light sources 30a, 30b, 32a, 32b, 34a, 34b, 36a, 36b, 38a, 38b, 40a, 40b in the different laser assemblies 12a, 12b may be different. Other variations are also possible. See, for example, U.S. Provisional Patent Application 63/403,925 titled “Device and Method for Non-Invasive Light Delivery to a Subject” filed Sep. 6, 2022, which is incorporated herein by reference in its entirety as well as the U.S. Provisional Application No. 63/450,215 titled “Non-Invasive, Dynamic, Multi-wavelength, Multi-node Photobiomodulation Therapy Methods and Systems for Treatment of Complex Diseases,” filed Mar. 6, 2023, which is also incorporated by reference herein in its entirety, and in particular, the Appendix of this provisional patent application, which includes U.S. Provisional Application No. 63/403,925, filed Sep. 6, 2022 to which non-provisional U.S. patent application Ser. No. 18/449,083, filed Aug. 14, 2023 as well as PCT Application No. PCT/CA2023/051076, filed Aug. 14, 2023 claim priority, which are also both incorporated by reference in their entirety, for additional discussion regarding devices for light delivery.

In various implementations, different light sources 30a, 30b, 32a, 32b, 34a, 34b, 36a, 36b, 38a, 38b, 40a, 40b may output different wavelength light than other of the light sources. For example, the first laser assembly 12a may include a first, second, third, fourth, fifth, and six light sources 30a, 32a, 34a, 36a, 38a, 40a having different output wavelength distributions. For example, the first light source 30a in the first assembly 12a may output light having a peak or central wavelength in the range from 780 to 810 nm, which is in the near infrared region. The second light source 32a in the first assembly 12a may output light having a peak or central wavelength in the range from 904 to 945 nm, which is also in the near infrared region. The third light source 34a in the first assembly 12a may output light having a peak or central wavelength in the range from 1,200 to 1,550 nm, which is in the mid infrared region. The fourth light source 36a in the first assembly 12a may output light having a peak or central wavelength in the range from 2,900 to 3,200 nm, which is in the far infrared region. The fifth light source 38a in the first assembly 12a may output light having a peak or central wavelength in the range from 630 to 700 nm, which is in the visible region. The sixth light source 40a in the first assembly 12a may output light having a peak or central wavelength in the range from 570 to 600 nm, which is also in the visible region. Other light sources that emit light in other ranges are also possible.

Similarly, the second laser assembly 12b may include a first, second, third, fourth, fifth, and six light sources 30b, 32b, 34b, 36b, 38b, 40b having different output wavelength distributions. For example, the first light source 30b in the second assembly 12b may output light having a peak or central wavelength in the range from 780 to 810 nm, which is in the near infrared region. The second light source 32b in the second assembly 12b may output light having a peak or central wavelength in the range from 904 to 945 nm, which is also in the near infrared region. The third light source 34b in the second assembly 12b may output light having a peak or central wavelength in the range from 1,200 to 1,550 nm, which is in the mid infrared region. The fourth light source 36b in the second assembly 12b may output light having a peak or central wavelength in the range from 2,900 to 3,200 nm, which is in the far infrared region. The fifth light source 38b in the second assembly 12b may output light having a peak or central wavelength in the range from 630 to 700 nm, which is in the visible region. The sixth light source 40b in the second assembly 12b may output light having a peak or central wavelength in the range from 570 to 600 nm, which is also in the visible region. Other light sources that emit light in other ranges are also possible. Thus, in various implementations of methods for treating a subject described herein, light of different wavelengths such as, for example, those discussed above, can be applied to the body 14.

As discussed above, the light from the light sources 30a, 30b, 32a, 32b, 34a, 34b, 36a, 36b, 38a, 38b, 40a, 40b may be modulated to produce optical pulses. In various implementations, the light from the light sources 30a, 30b, 32a, 32b, 34a, 34b, 36a, 36b, 38a, 38b, 40a, 40b may be modulated to produce pulses having a repetition rate in a range from 1 MHz to 15 MHz, possibly larger or smaller.

In some cases, different light sources 30a, 30b, 32a, 32b, 34a, 34b, 36a, 36b, 38a, 38b, 40a, 40b may output light having different repetition rates than light from other of the light sources. For example, different light sources 30a, 32a, 34a, 36a, 38a, 40a in the first laser assembly 12a may output light having different repetition rates than light from other of the light sources. For example, the first light source 30a in the first assembly 12a may output light having a repetition rate in the range from 9.5 to 10 MHz. The second light source 32a in the first assembly 12a may output light having a repetition rate in the range from 3 to 3.5 MHz. The third light source 34a in the first assembly 12a may output light having a repetition rate in the range from 6 to 6.5 MHz. The fourth light source 36a in the first assembly 12a may output light having a repetition rate in the range from 7 to 7.5 MHz. The fifth light source 38a in the first assembly 12a may output light having a repetition rate in the range from 4 to 4.5 MHz. The sixth light source 40a in the first assembly 12a may output light having a repetition rate in the range from 5 to 5.5 MHz. Although the different light sources 30a, 32a, 34a, 36a, 38a, 40a in the first laser assembly 12a have different repetition rates, in some implementations, one or more of the light sources have the same repetition rate. Other variations in the modulation rates as well as other modulation rates are also possible.

Similarly, the first light source 30b in the second assembly 12b may output light having a repetition rate in the range from 9.5 to 10 MHz. The second light source 32b in the second assembly 12b may output light having a repetition rate in the range from 3 to 3.5 MHz. The third light source 34b in the second assembly 12b may output light having a repetition rate in the range from 6 to 6.5 MHz. The fourth light source 36b in the second assembly 12b may output light having a repetition rate in the range from 7 to 7.5 MHz. The fifth light source 38b in the second assembly 12b may output light having a repetition rate in the range from 4 to 4.5 MHz. The sixth light source 40b in the second assembly 12b may output light having a repetition rate in the range from 5 to 5.5 MHz. Although the different light sources in the laser assembly 12b have different repetition rates, in some implementations, one or more of the light sources 30b, 32b, 34b, 36b, 38b, 40b have the same repetition rate. Other variations in the modulation rates as well as other modulation rates are also possible. Thus, in various implementations of methods for treating a subject described herein, the light applied to the body 14 may comprise pulses of light such as described above.

As discussed above, in various implementations one or more light sources 30a, 30b, 32a, 32b, 34a, 34b, 36a, 36b, 38a, 38b, 40a, 40b comprise laser diodes. Likewise, in various implementations the light source, laser, laser diode, or any combination thereof may be modulated, for example, using modulation and/or driver circuitry or electronics. Other approaches to modulating the light are possible including, for example, use of one or more optical modulators downstream of the light source 30a, 30b, 32a, 32b, 34a, 34b, 36a, 36b, 38a, 38b, 40a, 40b.

As discussed above light from the light source 30a, 30b, 32a, 32b, 34a, 34b, 36a, 36b, 38a, 38b, 40a, 40b may be coupled by optical fiber, e.g., optical fiber bundles to target areas of the body 14. In various implementations, the irradiance of light output from the light source, the optical fiber/optical fiber bundle, directed onto the subject (e.g., the skin of the subject) or any combination thereof is 20-100 mW, however, the irradiance can be larger or smaller. In some implementations, the modulation can have a duty cycle that is from 40-50%, however, a duty cycle that is larger or smaller may be employed. In some implementations the energy density of the light output from the light source, the optical fiber/optical fiber bundle, directed onto the subject (e.g., the skin of the subject) or any combination thereof is 0.66-1.5 J/cm², however, the energy density can be larger or smaller. In various designs, energy is delivered by one or more nodes or terminals comprising optic fibers and/or other delivery systems with a diameter or width of from 5-10 mm, although larger or smaller beam cross-sections are possible. Thus, in various implementations of methods for treating a subject described herein, light having one or more of the parameters discussed above can be applied to the body 14.

In the example shown in FIG. 1, the first laser assembly 12a is optically coupled to six optical fiber bundles 16a, 18a, 20a, 22a, 24a, 26a having distal ends configured (e.g., positioned) to direct light onto six different target areas, in this case on the subject's right side. In various implementations, these target areas are proximal to arteries such as the skin above the artery or within a range of 70, 60, 50, 40, 30, 20, 10, 5, 1 mm of the location on the skin just above the right carotid artery or any range formed by any of these values. For example, the distal end of the first optical fiber bundle 16a coupled to the first laser assembly 12a is configured (e.g., positioned and/or oriented) to direct light into the right carotid artery. The distal end of this first optical fiber bundle 16a may, therefore, be located within 100, 75, 50, 30, 20, 10, 5, 1 mm of the location on the skin just above the right carotid artery or any range formed by any of these values, and/or the distal end of the first optical fiber bundle 16a coupled to the first laser assembly 12a may be configured to direct light from the first light source 30a (or any other light source 32a, 34a, 36a, 38a, 40a in the laser assembly) to a location on the skin within 70, 60, 50, 30, 20, 10, 5, 1 mm of the location on the skin just above the right carotid artery or any range formed by any of these values. For example, optical fibers in the optical fiber bundle 16a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 16a to the target area. Likewise, in various implementations of methods described herein light is applied to a location proximal the right carotid artery such as to skin within 70, 60, 50, 30, 20, 10, 5, 1 mm of the location on the skin just above the right carotid artery or any range formed by any of these values.

Similarly, the distal end of the second optical fiber bundle 18a coupled to the first laser assembly 12a may be configured (e.g., positioned and/or oriented) to direct light into the right axillary artery. The distal end of this second optical fiber bundle 18a may, therefore, be located within 100, 75, 50, 30, 20, 10, 5, 1 mm of the location on the skin just above the right axillary artery or any range formed by any of these values, and/or the distal end of the second optical fiber 18a coupled to the first laser assembly 12a may be configured to direct light from the second light source 32a (or any other light source 30a, 34a, 36a, 38a, 40a in the laser assembly) to a location on the skin within 70, 60, 50, 30, 20, 10, 5, 1 mm of the location on the skin just above the right axillary artery or any range formed by any of these values. For example, optical fibers in the optical fiber bundle 18a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 18a to the target area. Likewise, in various implementations of methods described herein light is applied to a location proximal the right axillary artery such as to skin within 70, 60, 50, 30, 20, 10, 5, 1 mm of the location on the skin just above the right axillary artery or any range formed by any of these values.

The distal end of the third optical fiber bundle 20a coupled to the first laser assembly 12a may be configured (e.g., positioned and/or oriented) to direct light into the right femoral artery. The distal end of this third optical fiber bundle 20a may, therefore, be located within 100, 75, 50, 30, 20, 10, 5, 1 mm of the location on the skin just above the right femoral artery or any range formed by any of these values, and/or the distal end of the third optical fiber 20a coupled to the first laser assembly 12a may be configured to direct light from the third light source 34a (or any other light source 30a, 32a, 36a, 38a, 40a in the laser assembly) to a location on the skin within 70, 60, 50, 30, 20, 10, 5, 1 mm of the location on the skin just above the right femoral artery or any range formed by any of these values. For example, optical fibers in the optical fiber bundle 20a may be connected to different respective light sources 30a, 32a, 34a, 36a, 38a, 40a. Depending on which light source 30a 32a, 34a, 36a, 38a, 40a is activated, light from the activated light source will be delivered via the fiber bundle 20a to the target area. Likewise, in various implementations of methods described herein light is applied to a location proximal the right femoral artery such as to skin within 70, 60, 50, 30, 20, 10, 5, 1 mm of the location on the skin just above the right femoral artery or any range formed by any of these values.

The distal end of the fourth optical fiber bundle 22a coupled to the first laser assembly 12a may be configured (e.g., positioned and/or oriented) to direct light into the right popliteal artery. The distal end of this fourth optical fiber bundle 22a may, therefore, be located within 100, 75, 50, 30, 20, 10, 5, 1 mm of the location on the skin just above the right popliteal artery or any range formed by any of these values, and/or the distal end of the fourth optical fiber bundle 22a coupled to the first laser assembly 12a may be configured to direct light from the fourth light source 36a (or any other light source 30a, 32a, 34a, 38a, 40a in the laser assembly) to a location on the skin within 70, 60, 50, 30, 20, 10, 5, 1 mm of the location on the skin just above the right popliteal artery or any range formed by any of these values. For example, optical fibers in the optical fiber bundle 22a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 22a to the target area. Likewise, in various implementations of methods described herein light is applied to a location proximal the right popliteal artery such as to skin within 70, 60, 50, 30, 20, 10, 5, 1 mm of the location on the skin just above the right popliteal artery or any range formed by any of these values.

The distal end of the fifth optical fiber 24a coupled to the first laser assembly 12a may be configured (e.g., positioned and/or oriented) to direct light into the right anterior tibial artery. The distal end of this fifth optical fiber bundle 24a may, therefore, be located within 100, 75, 50, 30, 20, 10, 5, 1 mm of the location on the skin just above the right anterior tibial artery or any range formed by any of these values, and/or the distal end of the fifth optical fiber bundle 24a coupled to the first laser assembly 12a may be configured to direct light from the fifth light source 38a (or any other light source 30a, 32a, 34a, 36a, 40a in the laser assembly) to a location on the skin within 70, 60, 50, 30, 20, 10, 5, 1 mm of the location on the skin just above the right anterior tibial artery or any range formed by any of these values. For example, optical fibers in the optical fiber bundle 24a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 24a to the target area. Likewise, in various implementations of methods described herein light is applied to a location proximal the right anterior tibial artery such as to skin within 70, 60, 50, 30, 20, 10, 5, 1 mm of the location on the skin just above the right anterior tibial artery or any range formed by any of these values.

The distal end of the sixth optical fiber bundle 26a coupled to the first laser assembly 12a may be configured (e.g., positioned and/or oriented) to direct light into the right dorsalis pedis artery. The distal end of this sixth optical fiber bundle 26a may, therefore, be located within 100, 75, 50, 30, 20, 10, 5, 1 mm of the location on the skin just above the right dorsalis pedis artery or any range formed by any of these values, and/or the distal end of the fifth optical fiber bundle 26a coupled to the first laser assembly 12a may be configured to direct light from the sixth light source 40a (or any other light source 30a, 32a, 34a, 36a, 38a, in the laser assembly) to a location on the skin within 70, 60, 50, 30, 20, 10, 5, 1 mm of the location on the skin just above the right dorsal pedis is artery or any range formed by any of these values. For example, optical fibers in the optical fiber bundle 26a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 26a to the target area. Likewise, in various implementations of methods described herein light is applied to a location proximal the right dorsalis pedis artery such as to skin within 70, 60, 50, 30, 20, 10, 5, 1 mm of the location on the skin just above the right dorsal pedis artery or any range formed by any of these values.

In the example shown in FIG. 1, the second laser assembly 12b is likewise optically coupled to six optical fiber bundles 16b, 18b, 20b, 22b, 24b, 26b having distal ends configured (e.g., positioned) to direct light onto six different target areas, in this case on the subject's left side. In various implementations, these target areas are also proximal to arteries such as the skin above the artery or within a range of 70, 60, 50, 40, 30, 20, 10, 5, 1 mm of the location on the skin just above the artery or any range formed by any of these values. In particular, in various implementations, these arteries are the counterpart arteries that light from the light sources 30a, 32a, 34a, 36a, 38a, 40a in the first laser assembly 12a is directed toward. (This approach of directing light to the same arteries on both the left and right sides of the body or bilateral symmetrical pairs of arteries may be referred to herein as symmetric application of light. Light having the same properties, e.g., wavelength and/or pulse modulation repetition rate, may be the same for the counterpart arteries.). For example, the distal end of the first optical fiber bundle 16b coupled to the second laser assembly 12b is configured (e.g., positioned and/or oriented) to direct light into the left carotid artery (whereas the first optical fiber 16a coupled to the first laser assembly 12a is directed toward the right carotid artery). The distal end of this first optical fiber bundle 16b may, therefore, be located within 100, 75, 50, 30, 20, 10, 5, 1 mm of the location on the skin just above the left carotid artery or any range formed by any of these values, and/or the distal end of the first optical fiber bundle 16b coupled to the second laser assembly 12b may be configured to direct light from the first light source 30b (or any other light source 32b, 34b, 36b, 38b, 40b in the laser assembly) in the second laser assembly to a location on the skin within 70, 60, 50, 30, 20, 10, 5, 1 mm of the location on the skin just above the left carotid artery or any range formed by any of these values. For example, optical fibers in the optical fiber bundle 16b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 16b to the target area. Likewise, in various implementations of methods described herein light is applied to a location proximal the left carotid artery such as to skin within 70, 60, 50, 30, 20, 10, 5, 1 mm of the location on the skin just above the left carotid artery or any range formed by any of these values.

Similarly, the distal end of the second optical fiber bundle 18b coupled to the second laser assembly 12b may be configured (e.g., positioned and/or oriented) to direct light into the left axillary artery (whereas the second optical fiber 18a coupled to the first laser assembly 12a is directed toward the right axillary artery). The distal end of this second optical fiber bundle 18b coupled to the second laser assembly 12b may, therefore, be located within 100, 75, 50, 30, 20, 10, 5, 1 mm of the location on the skin just above the left axillary artery or any range formed by any of these values, and/or the distal end of the second optical fiber bundle 18b coupled to the second laser assembly 12b may be configured to direct light from the second light source 32b in the second laser assembly (or any other light source 30b, 34b, 36b, 38b, 40b in the laser assembly) to a location on the skin within 70, 60, 50, 30, 20, 10, 5, 1 mm of the location on the skin just above the axillary artery or any range formed by any of these values. For example, optical fibers in the optical fiber bundle 18b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 18b to the target area. Likewise, in various implementations of methods described herein light is applied to a location proximal the left axillary artery such as to skin within 70, 60, 50, 30, 20, 10, 5, 1 mm of the location on the skin just above the left axillary artery or any range formed by any of these values.

The distal end of the third optical fiber bundle 20b coupled to the second laser assembly 12b may be configured (e.g., positioned and/or oriented) to direct light into the left femoral artery (whereas the third optical fiber 20a coupled to the first laser assembly 12a is directed toward the right femoral artery). The distal end of this third optical fiber bundle 20b coupled to the second laser assembly 12b may, therefore, be located within 100, 75, 50, 30, 20, 10, 5, 1 mm of the location on the skin just above the left femoral artery or any range formed by any of these values, and/or the distal end of the third optical fiber bundle 20b coupled to the second laser assembly 12b may be configured to direct light from the third light source 34b in the second laser assembly 12b (or any other light source 30b, 32b, 36b, 38b, 40b in the laser assembly) to a location on the skin within 70, 60, 50, 30, 20, 10, 5, 1 mm of the location on the skin just above the left femoral artery or any range formed by any of these values. For example, optical fibers in the optical fiber bundle 20b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 20b to the target area. Likewise, in various implementations of methods described herein light is applied to a location proximal the left femoral artery such as to skin within 70, 60, 50, 30, 20, 10, 5, 1 mm of the location on the skin just above the left femoral artery or any range formed by any of these values.

The distal end of the fourth optical fiber bundle 22b coupled to the second laser assembly 12b may be configured (e.g., positioned and/or oriented) to direct light into the left popliteal artery (whereas the fourth optical fiber 22a coupled to the first laser assembly 12a is directed toward the right popliteal artery). The distal end of this fourth optical fiber bundle 22b coupled to the second laser assembly 12b may, therefore, be located within 100, 75, 50, 30, 20, 10, 5, 1 mm of the location on the skin just above the left popliteal artery or any range formed by any of these values, and/or the distal end of the fourth optical fiber bundle 22b coupled to the second laser assembly 12b may be configured to direct light from the fourth light source 36b in the second laser assembly 12b (or any other light source 30b, 32b, 34b, 38b, 40b in the laser assembly) to a location on the skin within 70, 60, 50, 30, 20, 10, 5, 1 mm of the location on the skin just above the left popliteal artery or any range formed by any of these values. For example, optical fibers in the optical fiber bundle 22b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 22b to the target area. Likewise, in various implementations of methods described herein light is applied to a location proximal the left popliteal artery such as to skin within 70, 60, 50, 30, 20, 10, 5, 1 mm of the location on the skin just above the left popliteal artery or any range formed by any of these values.

The distal end of the fifth optical fiber bundle 24b coupled to the second laser assembly 12b may be configured (e.g., positioned and/or oriented) to direct light into the left anterior tibial artery (whereas the fifth optical fiber 24a coupled to the first laser assembly 12a is directed toward the right tibial artery). The distal end of this fifth optical fiber 24b coupled to the second laser assembly 12b may, therefore, be located within 100, 75, 50, 30, 20, 10, 5, 1 mm of the location on the skin just above the left anterior tibial artery or any range formed by any of these values, and/or the distal end of the fifth optical fiber bundle 24b coupled to the second laser assembly 12b may be configured to direct light from the fifth light source 38b in the second laser assembly 12b (or any other light source 30b, 32b, 34b, 36b, 40b in the laser assembly) to a location on the skin within 70, 60, 50, 30, 20, 10, 5, 1 mm of the location on the skin just above the left anterior tibial artery or any range formed by any of these values. For example, optical fibers in the optical fiber bundle 24b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 24b to the target area. Likewise, in various implementations of methods described herein light is applied to a location proximal the left anterior tibial artery such as to skin within 70, 60, 50, 30, 20, 10, 5, 1 mm of the location on the skin just above the left anterior tibial artery or any range formed by any of these values.

The distal end of the sixth optical fiber bundle 26b coupled to the second laser assembly 12b may be configured (e.g., positioned and/or oriented) to direct light into the left dorsalis pedis artery (whereas the sixth optical fiber bundle 26a coupled to the first laser assembly 12a is directed toward the right dorsalis pedis artery). The distal end of this sixth optical fiber bundle 26b coupled to the first laser assembly 12b may, therefore, be located within 100, 75, 50, 30, 20, 10, 5, 1 mm of the location on the skin just above the left dorsalis pedis artery or any range formed by any of these values, and/or the distal end of the sixth optical fiber bundle 26b coupled to the second laser assembly 12b may be configured to direct light from the sixth light source 40b (or any other light source 30b, 32b, 34b, 36b, 38b in the laser assembly) to a location on the skin within 70, 60, 50, 30, 20, 10, 5, 1 mm of the location on the skin just above the dorsalis pedis artery or any range formed by any of these values. For example, optical fibers in the optical fiber bundle 26b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 26b to the target area. Likewise, in various implementations of methods described herein light is applied to a location proximal the left dorsalis pedis artery such as to skin within 70, 60, 50, 30, 20, 10, 5, 1 mm of the location on the skin just above the left dorsalis pedis artery or any range formed by any of these values.

Other configurations, however, are possible. For example, although FIG. 1 shows first and second laser assemblies 12a, 12b for right and left sides of the subject, alternative configurations are possible. More or less laser assemblies 12a, 12b, may be employed. For example, a single laser assembly can be used for each node, target location, (e.g., artery) to which light is to be applied. A plurality of fiber bundles can be included to propagate light produced by the laser assemblies to the subject. Each fiber bundle can extend from and be operatively connected to a corresponding one of the laser assemblies. Specifically, a proximal end of each fiber bundle can be connected to a corresponding one of the laser assemblies. Each fiber bundle can be formed from a plurality of optical fibers operatively connected to the corresponding laser assembly. A distal end of each fiber bundle can be arranged and configured for delivering light from the optical fibers to the subject, the distal end of the fiber bundle being formed at least in part by a distal end of each of the optical fibers. The distal end of each fiber bundle may include, for example, an adhesive pad connected thereto. The adhesive pad can be applied to the subject at a position to deliver light according to the light production program. As is mentioned above, the fiber bundle of each laser assembly can be formed from a plurality of optical fibers. Each optical fiber can be operatively connected to a corresponding one of the light sources (e.g., laser diodes) in the laser assembly, with a proximal end of each fiber receiving light from its corresponding light source (e.g., diode). See, for example, U.S. Patent Application 63/403,925 titled “Device and Method for Non-Invasive Light Delivery to a Subject” filed Sep. 6, 2022, which is incorporated herein by reference in its entirety for additional discussion regarding devices for light delivery as well as U.S. Provisional Application No. 63/450,215 titled “Non-Invasive, Dynamic, Multi-wavelength, Multi-node Photobiomodulation Therapy Methods and Systems for Treatment of Complex Diseases,” filed Mar. 6, 2023, which is also incorporated by reference herein in its entirety, and in particular, the Appendix of this provisional patent application, which includes U.S. Provisional Application No. 63/403,925, filed Sep. 6, 2022 to which non-provisional U.S. patent application Ser. No. 18/449,083, filed Aug. 14, 2023 as well as PCT Application No. PCT/CA2023/051076, filed Aug. 14, 2023 claim priority, which are also both incorporated by reference in their entirety. See also U.S. Pat. No. 5,231,984 to Lasb Laser Corp., issued Aug. 3, 1993.

FIG. 1 additionally shows control electronics 50 (or controller) that can be used, for example, to switch light sources 30a, 32a, 34a, 36a, 38a, 40a/30b, 32b, 34b, 36b, 38b, 40b on or off and/or to provide for modulation of light sources. In various designs, the control electronics 50 can provide for control of the execution of a regimen or program involving application of light of different (e.g., central) wavelength potentially for different durations, in different sessions, on different days or any one or more of these parameters. In some implementation, the control electronics 50 provide and/or select such a regimen or program.

Additionally, as illustrated in FIG. 1, in some designs, the control electronics 50 may be included in a separate location from the laser assemblies 12a, 12b although the control electronics may additionally be included within one or both laser assemblies 12a, 12b. The control electronics 50 may be solely or partly included within one or both of the laser assemblies 12a, 12b or may be partly or fully outside one or both of the laser assemblies. Accordingly, the control electronics 50 may be distributed over a plurality of locations including possibly one or both of the laser assemblies 12a, 12b and/or one or more locations outside the laser assemblies. Control electronics 50 may be located remotely from the system 10 or otherwise instructions may be provided remotely from the system 10.

The control electronics 50 (possibly included in one or more of the laser assemblies 12a, 12b or elsewhere) may be configured to select and/or control which light source(s) 30a, 32a, 34a, 36a, 38a, 40a/30b, 32b, 34b, 36b, 38b, 40b outputs light to the plurality of optical fibers or fiber bundles 16a, 18a, 20a, 22a, 24a, 26a/16b, 18b, 20b, 22b, 24b, 26b and/or is directed to the respective target areas at a given point in time. Such control electronics 50 may additionally sequence through a series of the light sources 30a, 32a, 34a, 36a, 38a, 40a/30b, 32b, 34b, 36b, 38b, 40b (e.g., in a given treatment session) such that light output by the optical fiber bundles 16a, 18a, 20a, 22a, 24a, 26a/16b, 18b, 20b, 22b, 24b, 26b and/or directed onto the subject at the target locations changes such that different wavelength light (having, e.g., a different central wavelength possibly with different pulse frequencies or repetition rates) are output at different times. One or more wavelengths previously used before a different wavelength is applied can be repeated again. Repetitions of more than one wavelength and/or multiple repetitions of the same wavelength or combinations thereof (e.g., in different parts of the sequence) are also possible. Other variations in the sequence of a session are possible.

The control electronics 50 (or controller) can also change the sequence in different sessions on the same or different days. The order of the sequence can be changed. More or less wavelengths (e.g., central wavelengths) can be added to or removed from the sequence, respectively. Repetition of the use of one or more wavelengths (e.g., central wavelengths) can be introduced and/or removed. Likewise, the number of times a light source or wavelength (e.g., central wavelengths) is included in the sequence can be altered.

Additionally, the individual durations that different wavelengths (e.g., central wavelengths) that are applied can be changed. The total duration over which light is applied in a session may also change, for example, with different sessions throughout a day. Similarly, such changes can be made on different days. For example, the number of sessions and/or the duration of the session can be changed and can be different for different days. The sequences used on different days can also be different.

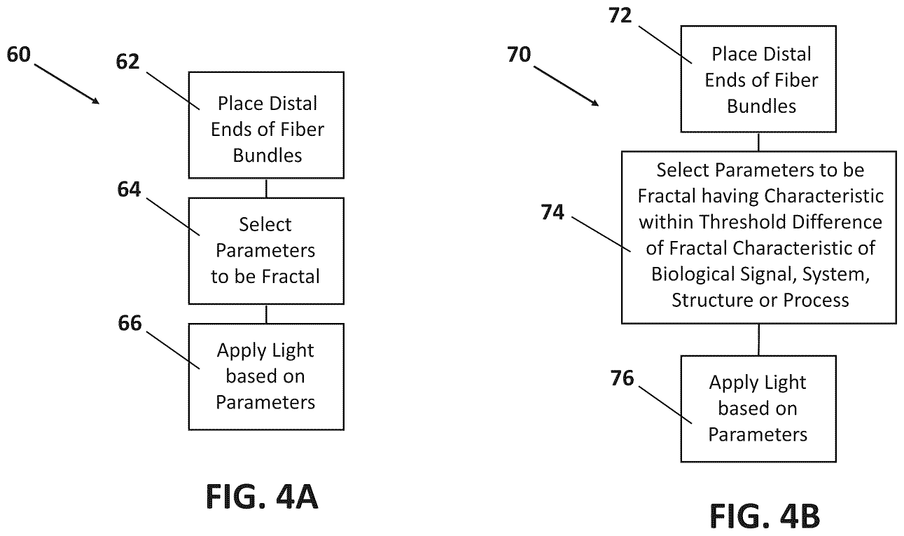

Accordingly, methods described herein may involve the application of light having peak or central wavelengths such as described above or elsewhere herein. This light may also be modulated, for example, to produce pulses, such as described above or elsewhere herein. Likewise, methods described herein may involve application of light to the locations described above or elsewhere herein. Thus, the light may be directed to locations proximal to arteries such as counterpart location such as counterpart arteries on left and right sides of the body and may be directed to within 100, 75, 50, 30, 20, 10, 5, 1 mm of the location on the skin just above the respective artery or any range formed by any of these values. Such light may, however, originate from different types of light sources and/or be delivered differently than shown in FIG. 1 and/or specifically discussed herein and thus need not necessarily use, for example, laser diodes and/or optical fibers or optical fiber bundles and/or the arrangement or configurations shown or described. Other light sources that emit light in other ranges are also possible. Light of other wavelengths (e.g., central wavelengths) and/or that is modulated differently than specifically described herein can be applied to the body 14 in various methods. Likewise, the number of locations to which light is applied may also be 2, 3, 4, 5, 6, 7, 8, 9, 10, 11, 12 or more or possibly less. The locations can also differ. For example, light may be directed to locations proximal other arteries. As described herein, the light may be applied in one or more sequences of optical pulses in one or more sessions on one or more days and have parameters such as duration, dosage (fractional dosage, accumulated dosage, etc.) or fluence, which may vary. The constituent light beams or signals and their characteristics and/or order of application of those light beams or signals or the different wavelengths (e.g., central wavelengths) of light having same or different pulse modulation repetition rates may also be also among the parameters, for example, in a regimen or program for treatment. Moreover, as discussed herein (e.g., in connection with FIG. 4A), one or more of these parameters and/or its variation with time or with respect to each other or one or more other variables may be a fractal, and/or have characteristics of a fractal. Likewise, in various implementations, one or more of these parameters may be selected, set, used, or changed, for example, by the control electronics 50, which may be in part or in total local and/or remote, so as to be a fractal and/or to have a characteristic of a fractal.

As additionally discussed herein (e.g., in connection with FIG. 4B), in some implementations, one or more of these parameters and or its variation with time or with respect to each other or one or more other variables may be a fractal and have characteristics of a fractal or otherwise have a characteristic of a fractal that corresponds to characteristic of a fractal in a biological signal, system, structure, or process. Similarly, in various implementations, one or more of these parameters may be selected, set, used, or changed, for example, by the control electronics 50 (or controller), which may be in part or in total local and/or remote, so as to be a fractal and have a characteristic of a fractal or otherwise have a characteristic of a fractal in a biological signal, system, structure, or process. For example, a plurality of parameters of the photobiomodulation method applied to a subject may be a fractal having a fractal characteristic (such as, e.g., a fractal dimension) that is the same or within a threshold difference (e.g., ±0.01%, ±0.1%, ±1%, ±5%, ±10%, ±20%, ±25% or any range formed by any of these values) of the fractal characteristic (e.g., a fractal dimension) of a biological signal, system, structure, or process. In some implementations, this biological signal, system, structure, or process that is a fractal or has a characteristic of a fractal may be the subject of or anatomical part or biological process for which the light treatment is being used.

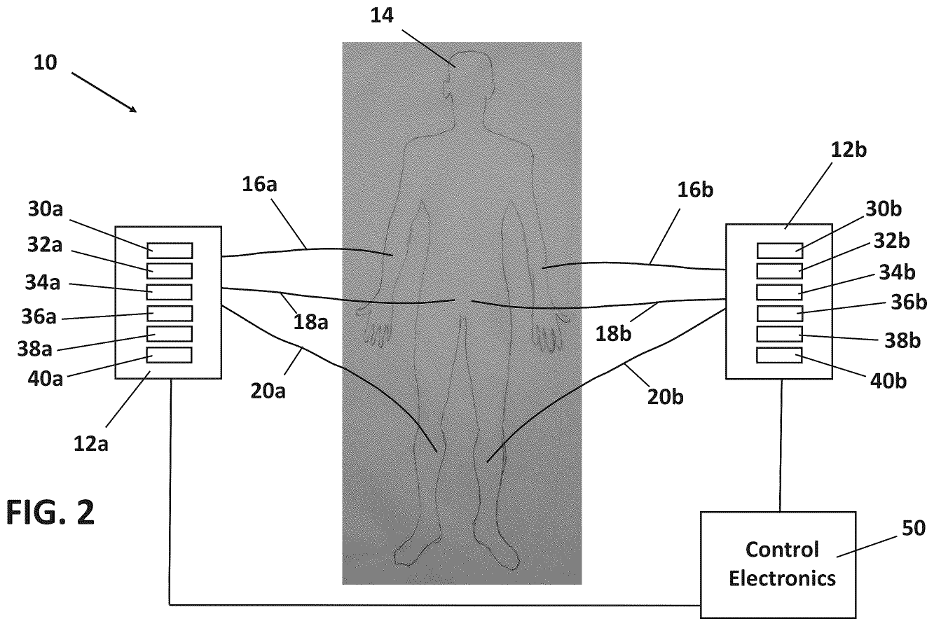

The locations where light is applied may be different. For example, the nodes or terminals may correspond to other locations on the body 14 as well. FIG. 2, for example, shows a system 10 comprising a plurality of optical fibers or optical fiber bundles 16a, 16b, 18a, 18b, 20a, 20b optically coupled to light sources 30a, 30b, 32a, 32b, 34a, 34b, 36a, 36b, 38a, 38b, 40a, 40b with first and second laser assemblies 12a, 12b where the distal ends of the optical fibers or optical fiber bundles are configured (e.g., positioned) to direct light to the left and right radial arteries, the left and right iliac arteries, and the left and right posterior tibial arteries of the subject. Likewise, in various implementations of methods described herein light is applied to locations proximal to one or more of the left and/and right radial arteries, the left and/or right iliac arteries, and the left and/or right posterior tibial arteries of the subject.

In the example shown in FIG. 2, the first laser assembly 12a is optically coupled to three optical fiber bundles 16a, 18a, 20a, having distal ends configured (e.g., positioned) to direct light onto three different target areas, in this case on the subject's right side. In various implementations, these target areas are proximal to arteries such as the skin above the artery or within a range of 70, 60, 50, 40, 30, 20, 10, 5, 1 mm of the location on the skin just above the right radial artery or any range formed by any of these values. For example, the distal end of the first optical fiber bundle 16a coupled to the first laser assembly 12a is configured (e.g., positioned and/or oriented) to direct light into the right radial artery. The distal end of this first optical fiber bundle 16a may, therefore, be located within 100, 75, 50, 30, 20, 10, 5, 1 mm of the location on the skin just above the right radial artery or any range formed by any of these values, and/or the distal end of the first optical fiber bundle 16a coupled to the first laser assembly 12a may be configured to direct light from the first light source 30a (or any other light source 32a, 34a, 36a, 38a, 40a in the laser assembly) to a location on the skin within 70, 60, 50, 30, 20, 10, 5, 1 mm of the location on the skin just above the right radial artery or any range formed by any of these values. For example, optical fibers in the optical fiber bundle 16a may be connected to different respective light sources 30a 32a, 34a, 36a, 38a, 40a. Depending on which light source 30a 32a, 34a, 36a, 38a, 40a is activated, light from the activated light source will be delivered via the fiber bundle 16a to the target area. Likewise, in various implementations of methods described herein light is applied to a location proximal the right radial artery such as to skin within 70, 60, 50, 30, 20, 10, 5, 1 mm of the location on the skin just above the right radial artery or any range formed by any of these values.

Similarly, the distal end of the second optical fiber bundle 18a coupled to the first laser assembly 12a may be configured (e.g., positioned and/or oriented) to direct light into the right iliac artery. The distal end of this second optical fiber bundle 18a may, therefore, be located within 100, 75, 50, 30, 20, 10, 5, 1 mm of the location on the skin just above the right iliac artery or any range formed by any of these values, and/or the distal end of the second optical fiber bundle 18a coupled to the first laser assembly 12a may be configured to direct light from the second light source 32a (or any other light source 30a, 34a, 36a, 38a, 40a in the laser assembly) to a location on the skin within 70, 60, 50, 30, 20, 10, 5, 1 mm of the location on the skin just above the right iliac artery or any range formed by any of these values. For example, optical fibers in the optical fiber bundle 18a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 18a to the target area. Likewise, in various implementations of methods described herein light is applied to a location proximal the right iliac artery such as to skin within 70, 60, 50, 30, 20, 10, 5, 1 mm of the location on the skin just above the right iliac artery or any range formed by any of these values.

The distal end of the third optical fiber bundle 20a coupled to the first laser assembly 12a may be configured (e.g., positioned and/or oriented) to direct light into the right posterior tibial artery. The distal end of this third optical fiber bundle 20a may, therefore, be located within 100, 75, 50, 30, 20, 10, 5, 1 mm of the location on the skin just above the right posterior tibial artery or any range formed by any of these values, and/or the distal end of the third optical fiber bundle 20a coupled to the first laser assembly 12a may be configured to direct light from the third light source 34a (or any other light source 30a, 32a, 36a, 38a, 40a in the laser assembly) to a location on the skin within 70, 60, 50, 30, 20, 10, 5, 1 mm of the location on the skin just above the right posterior tibial artery or any range formed by any of these values. For example, optical fibers in the optical fiber bundle 20a may be connected to different respective light sources 30a, 32a, 34a, 36a, 38a, 40a. Depending on which light source 30a, 32a, 34a, 36a, 38a, 40a is activated, light from the activated light source will be delivered via the fiber bundle 20a to the target area. Likewise, in various implementations of methods described herein light is applied to a location proximal the right posterior tibial artery such as to skin within 70, 60, 50, 30, 20, 10, 5, 1 mm of the location on the skin just above the right posterior tibial artery or any range formed by any of these values.

In the example shown in FIG. 2, the second laser assembly 12b is likewise optically coupled to three optical fiber bundles 16b, 18b, 20b having distal ends configured (e.g., positioned) to direct light onto three different target areas, in this case on the subject's left side. In various implementations, these target areas are also proximal to arteries such as the skin above the artery or within a range of 70, 60, 50, 40, 30, 20, 10, 5, 1 mm of the location on the skin just above the artery or any range formed by any of these values. In particular, in various implementations, these arteries are the counterpart arteries that light from the light sources 30a, 32a, 34a, 36a, 38a, 40a in the first laser assembly 12a is directed toward. (This approach of directing light to the same arteries on both the left and right sides of the body or bilateral symmetrical pairs of arteries may be referred to herein as symmetric application of light. Light having the same properties, e.g., wavelength (e.g., central wavelengths) and/or pulse modulation repetition rate, may be the same for the counterpart arteries.) For example, the distal end of the first optical fiber bundle 16b coupled to the second laser assembly 12b is configured (e.g., positioned and/or oriented) to direct light into the left radial artery (whereas the first optical fiber bundle 16a coupled to the first laser assembly 12a is directed toward the right radial artery). The distal end of this first optical fiber bundle 16b may, therefore, be located within 100, 75, 50, 30, 20, 10, 5, 1 mm of the location on the skin just above the left radial artery or any range formed by any of these values, and/or the distal end of the first optical fiber bundle 16b coupled to the second laser assembly 12b may be configured to direct light from the first light source 30b (or any other light source 32b, 34b, 36b, 38b, 40b in the laser assembly) in the second laser assembly to a location on the skin within 70, 60, 50, 30, 20, 10, 5, 1 mm of the location on the skin just above the left radial artery or any range formed by any of these values. For example, optical fibers in the optical fiber bundle 16b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 16b to the target area. Likewise, in various implementations of methods described herein light is applied to a location proximal the left radial artery such as to skin within 70, 60, 50, 30, 20, 10, 5, 1 mm of the location on the skin just above the left radial artery or any range formed by any of these values.

Similarly, the distal end of the second optical fiber bundle 18b coupled to the second laser assembly 12b may be configured (e.g., positioned and/or oriented) to direct light into the left iliac artery (whereas the second optical fiber bundle 18a coupled to the first laser assembly 12a is directed toward the right iliac artery). The distal end of this second optical fiber bundle 18b coupled to the second laser assembly 12b may, therefore, be located within 100, 75, 50, 30, 20, 10, 5, 1 mm of the location on the skin just above the left iliac artery or any range formed by any of these values, and/or the distal end of the second optical fiber bundle 18b coupled to the second laser assembly 12b may be configured to direct light from the second light source 32b (or any other light source 30b, 34b, 36b, 38b, 40b in the laser assembly) in the second laser assembly to a location on the skin within 70, 60, 50, 30, 20, 10, 5, 1 mm of the location on the skin just above the left iliac artery or any range formed by any of these values. For example, optical fibers in the optical fiber bundle 18b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 18b to the target area. Likewise, in various implementations of methods described herein light is applied to a location proximal the left iliac artery such as to skin within 70, 60, 50, 30, 20, 10, 5, 1 mm of the location on the skin just above the left iliac artery or any range formed by any of these values.

The distal end of the third optical fiber bundle 20b coupled to the second laser assembly 12b may be configured (e.g., positioned and/or oriented) to direct light into the left posterior tibial artery (whereas the third optical fiber bundle 20a coupled to the first laser assembly 12a is directed toward the right posterior tibial artery). The distal end of this third optical fiber bundle 20b coupled to the second laser assembly 12b may, therefore, be located within 100, 75, 50, 30, 20, 10, 5, 1 mm of the location on the skin just above the left posterior tibial artery or any range formed by any of these values, and/or the distal end of the third optical fiber bundle 20b coupled to the second laser assembly 12b may be configured to direct light from the third light source 34b (or any other light source 30b, 32b, 36b, 38b, 40b in the laser assembly) in the second laser assembly 12b to a location on the skin within 70, 60, 50, 30, 20, 10, 5, 1 mm of the location on the skin just above the left posterior tibial artery or any range formed by any of these values. For example, optical fibers in the optical fiber bundle 20b may be connected to different respective light sources 30b, 32b, 34b, 36b, 38b, 40b. Depending on which light source 30b, 32b, 34b, 36b, 38b, 40b is activated, light from the activated light source will be delivered via the fiber bundle 20b to the target area. Likewise, in various implementations of methods described herein light is applied to a location proximal the left posterior tibial artery such as to skin within 70, 60, 50, 30, 20, 10, 5, 1 mm of the location on the skin just above the left posterior tibial artery or any range formed by any of these values.

As illustrated in FIG. 2, the number of nodes or terminals and/or target locations may vary. In some implementations, for example, the number of nodes and/or target regions may be 2, 3, 4, 5, 6, 7, 8, 9, 10, 11, 12, or any range formed by any of these values or possibly larger. The number of nodes and/or target regions need not be equal to the number of light sources of different wavelength (e.g., central wavelength). The number of light sources having different wavelength (e.g., different central wavelength), however, may also vary. The number of light sources, for example, may be 1, 2, 3, 4, 5, 6, 7, 8, 9, 10 or any range between any of these values and may be larger.

Accordingly, light may be applied to a range of bilateral symmetrical pairs including but not limited to the left and right carotid arteries, the left and right axillary arteries, the left and right femoral arteries, the left and right popliteal arteries, the left and right tibial arteries, the left and right dorsalis pedis arteries, the left and right radial arteries, the left and right iliac arteries, or the left and right posterior tibial arteries or any combination of these. In various implementation, light is applied to at least 1, 2, 3, 4, 5, 6, 7, 8, 9, 10 of the bilateral symmetrical pairs listed above or to any range between any of these values. Light may additionally be applied to other bilateral symmetrical pairs not listed here. In various implementations, the light having the same characteristics, for example, having the same wavelength (e.g., central wavelength) and/or pulse repetition rate, is directed to different locations such as different arteries simultaneously or at the same time. In various implementations, light having the same characteristics, for example, having the same wavelength (e.g., central wavelength) and/or pulse repetition rate, is directed to counterpart locations such as counterpart arteries simultaneously or at the same time. Likewise, in various implementations, this light is simultaneously applied for the same duration to different locations, e.g., different arteries and/or to same counterpart locations such as the same counterpart arteries.

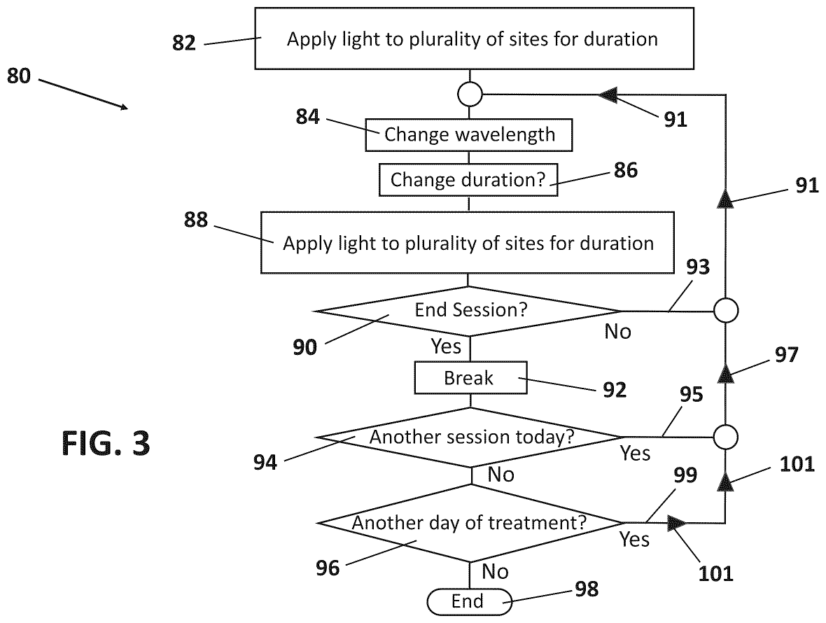

FIG. 3 comprises a block diagram 80 illustrating a method for producing and/or executing a regimen of applying light to a plurality of sites on a subject to provide medical treatment or therapy. As illustrated in block 82, the regimen involves applying light to a plurality of sites or target areas for a duration of time. This light has a wavelength such as a peak or central wavelength. This light may be modulated, for example, to produce optical pulses as described herein, and may have a corresponding pulse frequency or repetition rate. After application of this light, light of another wavelength (e.g., central wavelength) can be applied as illustrated by blocks 84 and 88. Block 84 represent changing the wavelength (e.g., central wavelength), while block 88 represents applying the light to the target locations. The duration during which this light is applied can be the same or can be different than the duration over which the previous light having a different wavelength (e.g., different central wavelength) was applied as represented by block 86. After the duration over which this light is applied, the session may end or light of a different wavelength (e.g., different central wavelength) can be applied to the target sites. Diamond 90 represents the option of ending the session or continuing with application of more light of a different wavelength (e.g., different central wavelength). In the later case, loop 93 and arrows 91 indicate that the continuation of the session involves changing the wavelength (e.g., central wavelength) (block 84), determining whether to change the duration (block 86) and applying the light as represented by block 88. The session can continue or end as indicated by the return to diamond 90. This loop 93 can be repeated as long as the session is to continue, further adding to the sequence of light signals being applied (e.g., light sources being activated and light being applied). When the session and sequence are to end, the process proceeds to block 92 where a break is taken. If another session is to be performed, at diamond 94, the process proceeds back along loop 95 as indicated by arrow 97 to block 84 where the wavelength (e.g., central wavelength) of light may be changed (although in some cases, the wavelength (e.g., central wavelength) need not change with the start of a new session) and the duration may or may not be changed. The process can continue repeating through loop 93 proceeding through the sequence until the session is completed and another break is taken at blocks 90 and 92. If another session is to be performed, at diamond 94 the process proceeds again back through loop 95 as indicated by arrow 97 to perform another session. If the session is the last session of the day, at diamond 94, the process proceeds to diamond 96. If the treatment is to continue on another day, the process proceeds along loop 99 as indicated by arrows 101 back to block 84 where the (e.g., central wavelength) may be changed (although the wavelength need not always be changed with a new day). If a plurality of sessions is to be completed, the process cycles through loop 93 and 95 as discussed above with a sequence for different respective sessions. If all the sessions are completed for that day, a determination is made at diamond 96 whether the treatment will continue for more days. The treatment can continue a number of days, possibly weeks, or longer following loop 99 as indicated by arrows 101. When the regimen is complete, with no more additional days of treatment, the process continues from diamond 96 to oval 98.

This flow diagram 80 represents one possible method of providing and/or executing a treatment regimen wherein light is applied to a plurality of sites on a subject. This process can be implemented to produce and/or execute a wide range of different regimens. In some cases, a session includes application a light of different wavelengths (e.g., different central wavelengths) at different times. Additionally, in some cases, the same wavelength (e.g., central wavelength) of light can be applied to the target sites at different time in the session. The duration over which a given wavelength (e.g., central wavelength) of light is applied can be changed or not.

Other methods may be employed to produce, provide and/or executed different regimens. Thus, this flow diagram may be changed or other flow diagrams may be used. Steps can be added, removed, rearranged, reordered, changed, or any combination of these. For example, the order of steps can be changed. One or more blocks can be removed. One or more blocks can be split up. Still other variations may be possible.

Accordingly, in some examples, optical pulses of a first wavelength (e.g., first central wavelength) and a first repetition rate may be applied to a plurality of anatomical locations on the body 14 for a first temporal duration and after applying the optical pulses of the first wavelength (e.g., first central wavelength), optical pulses of a second wavelength different (e.g., second central wavelength) than the first wavelength and having a second repetition rate are applied to the plurality of anatomical locations on the body 14 for a second temporal duration. In some implementations, the second repetition rate is different than the first repetition rate, however the first repetition rate could be the same as the second repetition rate. Additionally, in some implementations, the second temporal duration is different than the first temporal duration, however the first temporal duration could be the same as the second temporal duration. In some implementations, the locations on the body are locations 14 on the skin proximal to arteries such as, for example, as described above. In some implementations, after applying the optical pulses of the second wavelength (e.g., second central wavelength), optical pulses of a third wavelength (e.g., third central wavelength) different than the second wavelength and having a third repetition rate are applied to the plurality of anatomical locations on the body 14 for a third temporal duration thereby adding to the sequence. Likewise, in some implementations, after applying the optical pulses of the third wavelength (e.g., third central wavelength), optical pulses of a fourth wavelength (e.g., fourth central wavelength) different than the third wavelength and having a fourth repetition rate are applied to the plurality of anatomical locations on the body 14 for a fourth temporal duration thereby adding to the sequence. Similarly, in some implementations, after applying the optical pulses of the fourth wavelength (e.g., fourth central wavelength), optical pulses of a fifth wavelength (e.g., fifth central wavelength) different than the fourth wavelength and a fifth repetition rate are applied to the plurality of anatomical locations on the body 14 for a fifth temporal duration thereby adding to the sequence. Additionally, in some implementations, after applying the optical pulses of the fifth wavelength (e.g., fifth central wavelength), optical pulses of a sixth wavelength (e.g., sixth central wavelength) different than the fifth wavelength and a sixth repetition rate are applied to the plurality of anatomical locations on the body 14 for a sixth temporal duration thereby adding to the sequence.

In various implementations, the first sequence can further comprise repeating application of optical pulses of one wavelength (e.g., one central wavelength). The reoccurrence of the optical pulses of the one wavelength can occur after optical pulses of a different wavelength (e.g., different central wavelength) have been applied after optical pulses of the one wavelength had been previously used in the sequence. Accordingly, the first sequence may return to the use of one wavelength (e.g., one central wavelength) after another wavelength (e.g., another central wavelength) has been used in the interim.

The sequences can also change with session. For example, in a second session after the first session on the same day, the duration of application of at least one of the wavelengths (e.g., central wavelengths) in the first sequence can be changed. Similarly, in a second session after a first session of an earlier day, respective durations of application of a plurality of the wavelengths (e.g., central wavelengths) in the sequence can be changed. Additionally, in a third session after the second session on the same day, the duration of application of at least one of the wavelengths (e.g., central wavelengths) in the second sequence can be changed with respect to that of the second session. Additionally, in a third session after the second session on the same day, respective durations of application of a plurality of the wavelengths (e.g., central wavelengths) in the second sequence can be changed with respect to the second session.

The sequence can also change with the day. For example, in various implementations, for a first session of a second day, the duration of application of at least one of the wavelengths (e.g., central wavelengths) in the first sequence can be changed with respect to the first session of the first day. Similarly, for a first session of the second day, respective durations of application of a plurality of the wavelengths (e.g., central wavelengths) in the first sequence changes with respect to the first session of the first day. Such a change can be implemented in multiple sessions on the same day as well. For example, for a second session on the second day, the duration of application of at least one of the wavelengths (e.g., central wavelengths) in the first sequence can be changed with respect to the first session of the first day and/or with respect to the first session of the second day. Additionally, for a second session of the second day, respective durations of application of a plurality of the wavelengths (e.g., central wavelengths) in the first sequence can be changed with respect to the first session of the first day and/or with respect to the first session of the second day.

The aggregate duration, which is the total duration that the pulsed light is applied regardless of the wavelength (e.g., central wavelength) or other parameter (e.g., repetition rate), can also be changed. For example, a plurality of optical pulses can be applied on a second day wherein the aggregate duration of the pulses of light applied on a second day has different duration than the application of the plurality of optical pulses on the first day. Likewise, the aggregate duration can change from one day to the next.

The number of sessions per day can also be varied. In some implementations, on the first day a plurality of pulses of light are applied in a plurality of sessions and on a second day a plurality of pulses of light are applied in a plurality of sessions, and the number of sessions on the second day is different than the number of sessions on the first day. Likewise, on a third day a plurality of pulses of light can be applied in a plurality of sessions, and the number of sessions on the third day can be different than the number of sessions on the second day.

In various implementations, the number of sessions on a given day is at least two session and may be larger. Also, as used herein, the term “first”, e.g., applying a first wavelength (e.g., first central wavelength), a first pulse modulation repetition rate, a first signal, a first light component, light from a first light source or a first optical fiber or first optical fiber bundle, a first sequence, a first session, a first day, etc., does not preclude other prior similar actions, step, events, item, element, component or group, etc., for example, application of one or more other wavelengths (e.g., central wavelengths), one or more other pulse modulation repetition rates, one or more other signals, one or more other light components, light from one or more other light sources or optical fibers or optical fiber bundles, one or more other sequences, one or more other sessions, one or more other days, etc., prior. Rather, the term first is used as a matter of convenience for identification of an action, step, event, item, element, component or group, etc.

Any combination of such features can be included in a regimen or program for light treatment. Many other changes or variations are also possible.

As referred to above, in various implementations, the parameters of the regimen or program or portion thereof are selected or set such that, or are otherwise such that, the variation of the parameters in the regimen or program comprises one or more fractals or exhibits one or more characteristics of one or more fractals. This method is depicted in the block diagram 60 shown in FIG. 4A. This method includes establishing the location of the nodes or terminal or where light is to be delivered, for example, placing the distal ends of the plurality of optical fibers or optical fiber bundles at the appropriate location as represented by block 62. As discussed herein, the location of where light is applied and/or the position of the terminals or nodes may be proximal to arteries of the body 14. The parameters of the treatment regimen or program are selected, determined, provided, set, or established as represented by block 64. Some possible example parameters include the wavelengths (e.g., central wavelengths), the frequency or repetition rate of the pulses in the modulated optical signal, the duration during which light of a particular wavelength (e.g., particular central wavelength) is applied, the fluence or dosage (e.g., fractional dosage and/or accumulated dosage), the order of the sequence of wavelengths (e.g., central wavelengths) applied (including possible repeats), the number of sessions per day (which may change for different days), the distribution of and/or variation of dosage or fluence in different portions of the sequence and/or different sessions and/or different days, the aggregate amount of light applied during a session, during multiple sessions throughout the day, for different days, etc. The parameters that comprise a regimen or program are not limited to these. In various implementations, a number of these parameters make-up a multi-dimensional space and these parameters may comprise fractals in that space. In various implementations, multiple parameters comprise a multifractal system. The regimen or program can be executed, for example, by applying light based on the parameters of the regimen or program as represented by block 66.

Other variations are possible. Steps can be added, removed, rearranged, reordered, changed, or any combination of these. For example, the order of steps can be changed. Block 64 directed to selecting, determining, providing, setting or establishing parameters to be fractal can be performed prior to block 62, which involve determining or establishing where to direct light, e.g., positioning the nodes, e.g., placing the distal ends of the fiber bundles. Blocks such as block 62 can be removed. Light can be simply directed to the desired particular locations in block 66. Additionally, one or more blocks can be split up. For example, block 64 comprising selecting parameters to be a fractal may involve steps prior to block 62 and steps after block 64 in some implementations and thus the block diagram 60 can change.

Moreover, in various implementations, the parameters of the regimen or program or portion thereof are selected or set such that, or are otherwise such that, the variation of the parameters in the regimen or program comprises one or more fractals or exhibits one or more characteristics of one or more fractal within a threshold difference (e.g., ±0.01%, ±0.1%, ±1%, ±5%, ±10%, ±20%, ±25% or any range formed by any of these values) of one or more fractals or characteristic of one or more fractals of a biological signal, structure, system or process as depicted in the method shown in a block diagram 70 in FIG. 4B.

This method may include establishing the location of the nodes or terminal and/or where light is to be delivered, for example, by placing the distal ends of the plurality of optical fibers or optical fiber bundles at the appropriate location as represented by block 72. As discussed herein, the location where light is applied and/or the position of the terminals or nodes may be proximal to arteries of the body 14. The parameters of the treatment regimen or program are selected, determined, provided, set, or established as represented by block 74. Some possible example parameters include the wavelengths (e.g., central wavelengths), the frequency or repetition rate of the pulses in the modulated optical signal, the duration during which light of a particular wavelength (e.g., particular central wavelength) is applied, the fluence or dosage (e.g., fractional dosage and/or accumulated dosage), the order of the sequence of wavelengths (e.g., central wavelengths) applied (including possible repeats), the number of sessions per day (which may change for different days), the distribution of and/or variation of dosage or fluence in different portions of the sequence and/or different sessions and/or different days, the aggregate amount of light applied during a session, during multiple sessions throughout the day, for different days, etc. The parameters that comprise a regimen or program are not limited to these. In various implementations, a number of these parameters make-up a multi-dimension space and these parameters may comprise fractals in that space. In various implementations, multiple parameters comprise a multifractal system. Moreover, the parameters of the regimen or program or portion thereof are selected or set such that, or are otherwise such that, the variation of the parameters in the regimen or program comprises one or more fractals or exhibits one or more characteristics of one or more fractals within a threshold difference (e.g., ±0.01%, ±0.1%, ±1%, ±5%, ±10%, ±20%, ±25% or any range formed by any of these values) of one or more fractals or characteristic of one or more fractals of a biological signal, structure, system or process as depicted in method shown in a block 74.

The regimen or program can be executed, for example, by applying light based on the parameters of the regimen or program as represented by block 76.

Other variations are possible. Steps can be added, removed, rearrange, reordered, changed, or any combination of these. For example, the order of steps can be changed. Block 74 directed to selecting, determining, providing, setting or establishing parameters to be fractal can be performed prior to block 72, which involve positioning the nodes, e.g., placing the distal ends of the fiber bundles. Additionally, one or more blocks can be split up. For example, block 74 comprising selecting parameters to be a fractal may involve steps prior to block 72 and steps after block 74 in some implementations and thus the block diagram can change.

Software and/or electronics (e.g., control electronics or controller) may be configured to select, determine, set, or provide a regimen or program of light treatment that includes one or more sequences of lights to be illuminated in different sessions on one or possibly a plurality of days. In some implementations, the software and/or electronics comprise and/or utilized algorithms and/or artificial intelligence and/or machine learning or any combination thereof to provide such a regimen or program. In some cases, the software and/or electronics and/or algorithms and/or artificial intelligence and/or machine learning, or any combination thereof dynamically configure the signal and control the fractioned and/or total dosage. In some cases, this signal is non-linear and may potentially be chaotic.

In some implementations, such software and/or electronics (control electronics or controller), which may possibly employ artificial intelligence and/or machine learning, may be configured to determine regimens or programs for photobiomodulation therapy or medical treatment using pulsed light in which one or more of the parameters of the regimen or program comprise one or more fractals or exhibit characteristics of one or more fractals as described above. Moreover, in some implementations, the software and/or electronics (e.g., control electronics or controller), which may possibly employ artificial intelligence and/or machine learning, may be configured to determine regimens or programs for photobiomodulation therapy or medical treatment using pulsed light in which one or more of the parameters of the regimen or program comprise one or more fractals or exhibit characteristics of one or more fractals within a threshold difference of one or more fractals and/or characteristics of fractal behavior exhibited by a biological signal system, structure, or process, which may or may not be the subject of the photobiomodulation therapy or medical treatment as described above.

Additionally, in various implementations, the system can include and/or the method may employ sensors to provide biofeedback and/or information regarding the body of the subject. Example sensors can include sensors configured to measure voltage, peripheral flow, oxygen concentration or other variables. Signal testers or monitors such as photodetectors and/or electronics can be configured to monitor the optical signals. In various implementations, the system and/or method can be configured to store and transmit data securely for treatment control and/or research purposes in accord with applicable laws. To enhance security, device operation and/or treatment (e.g., application of the light to the subject's body) can be subject to security safeguards and/or controls such as biometric sensors configured to identify and/or confirm the identity of one or more authorized users.

As discussed above, a method for treating a subject with pulsed light may utilized a non-invasive, dynamic, multi-frequency, multi-node photobiomodulation therapy system comprising multiple light sources or laser diodes. These light sources may have different wavelength (e.g., central wavelength). For example, these light sources may comprise the following types of laser diodes:
Near infrared: 780-810 nm wavelength output pulsed at 9.5-10 MHz
Near infrared: 904-945 nm wavelength output pulsed at 3-3.5 MHz
Mid infrared: 1200-1,550 nm wavelength output pulsed at 6-6.5 MHz
Far infrared: 2,900-3,200 nm wavelength output pulsed at 7-7.5 MHz
Red: 630-700 nm wavelength output pulsed at 4-4.5 MHz; or
Yellow: 570-600 nm wavelength output pulsed at 5-5.5 MHz.

The different laser diodes may be configured to output a peak power in the range of from 25-100 mW in some configurations. The laser diodes may be configured to provide a pulse duration of 200 ns. The laser diodes may be configured to output a modulated or pulsed output signal having a duty cycle from 40-50%. In some configurations, a plurality of diodes are grouped into blocks or arrays. In some designs, the laser diodes are configured to operate in a non-linear (possibly chaotic) regime.

In some designs and/or methods, energy is simultaneously delivered to one or more nodes or terminals and/or target locations of the body of the subject such as on skin proximal to arteries. In some designs and/or methods, optical fibers, optical fiber bundles and/or other optical systems may be configured or used to deliver the light to the target areas. In some implementations, a beam is directed to the target areas wherein the beam has a 5-10 mm cross-sectional width or diameter.

Accordingly, in various example implementations described herein, the optical signal comprises infrared or visible light with a specific wavelength (e.g., specific central wavelength) and an amplitude that is modulated, e.g., pulsed, at a MegaHertz or ultrasonic frequency (carrier wave). In various implementations, the different signal components having different wavelengths (e.g., different central wavelengths) are pulsed in intensity or amplitude, which can give a unique identity to the different wavelength component signals if the modulations are different and/or the amplitudes of the pulses are different for different wavelength signal components. In some implementations, some other photonic variables such as wave shape, duty cycle, duration of the pulse, etc. remain constant for the different signals. In some cases, the duration of the application of the light of a particular wavelength (e.g., particular central wavelengths) varies. In some implementations, as described above, the sequence or order in which the signal components are arranged or organized for different treatments, the duration of their ON and OFF times, their distribution thorough multiple sessions, e.g., in a 12-hour window period of a day, or any combination of these and possibly some other parameters follow a fractal dimension morphology. In some implementations, the sequences change with different sessions and/or on different days and such changing sequences may comprise different (e.g., continuous) fractals or multiple fractals or may provide for a multifractal system. The on/off signal sequence may follow a variable temporal fractal dimension. Such fractal dimension can also be understood as a non-linear, fractional signal. A daily fractioned dose and/or an accumulated fractioned dose may also be based on a multifractal system.

Additionally, the optical signal itself may be a fractal, comprise one or more fractals, or exhibit one or more fractal properties or characteristics. In some implementations, the switching between different wavelength light sources in the sequence may be sufficiently fast such the optical signal produced by the plurality of light sources used in the sequence may comprise one or more fractals or have features of one or more fractals that are within a threshold difference (e.g., ±0.01%, ±0.1%, ±1%, ±5%, ±10%, ±20%, ±25% or any range formed by any of these values) of features of one or more fractals in the biological signal, system, structure or process. The time-space fractals, for example, can span the range from micro to meso to macro scales. Accordingly, multiple frequencies can match or be within a threshold difference of the fractal, fractal or multifractal properties of the biological signal, system, structure, or process are possible.

One or more of these and possibly other of the parameters may be configured to produce a multifractal system.

Accordingly, in some implementations, the sequence of light signals results in a space time geometry or in the parameter versus time space that is a fractal. In various implementations, the fractal discussed herein is a strange fractal. In some cases, the fractal may be one based on the Henon attractor or the Lorenz attractor. In various implementations some of the parameters may be characterized by log functions, exponential functions, exponential functions with a fraction as the exponent or any combination thereof.

Accordingly, as discussed above, parameters such as the locations, duration, number of sessions, dosage (e.g., fractional dosage and/or accumulated dosage), and other aspects of the method, regimen, program, as variables in a mathematical system, may be fractal in nature, and/or meet the characteristics of a fractal system such as for example having symmetry of origin, and/or self-similarity at different scales or be fractals. These parameters may form multiple fractals and/or a multifractal system or contribute to producing a multifractal system. Moreover, as described above, the fractal nature of the signal and/or regimen resulting from the application of this method may replicate and couple with the fractal characteristics of the biological system (e.g., the vascular, respiratory system, central nervous system and peripheral nerves, among others).

Numerous biological signals, systems, components, and processes, such as in a human or animal body are fractal in nature. The spatial distribution of the nerves in the nervous system or blood vessels in the circulatory system, the temporal variation of signals such as electrical signals from the heart, brainwaves, etc. as well as biochemical processes may comprise fractals or exhibit characteristics of fractals.

Accordingly, as discussed above, in various implementations described herein, the application of optical signals to the body 14, for example, the parameters, the regimen, the signals, or any combination thereof, comprise a fractal, multiple fractals, (a) multifractal system(s), and/or exhibit characteristics of a fractal, multiple fractals, multifractal systems that are within a threshold difference (e.g., ±0.01%, ±0.1%, ±1%, ±5%, ±10%, ±20%, ±25% or any range formed by any of these values) of the fractal, multiple fractals, multifractal system(s), and/or the properties exhibited by or characteristics of the fractal, multiple fractals, multifractal systems of the signals, system, structures, and processes of the body. In various implementations, for example, coupling occurs between the functional fractal structure of the signal and the functional fractal structure of the biological system as a result of sufficiently matching the functional fractal structure of the signal and the functional fractal structure of the biological system or parameters and/or characteristics thereof. As discussed above, for example, the sequence of signals describes morphologic structures that reproduce fractal geometries. Different elements that are part of the signal may be structured to create a fractal dimension, which may be represented by an algorithm. Parameters of the regimen may be fractals.

In various implementations, the biological signal, system, structure, or process that comprises a fractal, multiple fractals, a multifractal system or exhibits characteristic of a fractal, multiple fractals, or a multifractal system derives from or comprises water in the body.

Accordingly, water may be utilized in various ways in methods involving non-invasive non-ionizing application of light to a subject for medical treatment or therapy such as those describe herein. In some methods described herein, water is used as a universal attractor. In some methods, water molecules are used as an extended rechargeable electrolytic biobattery.

A wide range of mechanisms, however, can be employed. In some implementations, transmission of radiant energy through the vascular system induces coupling of photic energies supplied with multiple ligands at different time-space scales that follow a dynamic system and intervene in the restitution of physiological rhythms. In some cases, for example, there is synchronization of multiple spacetime ligands even though their coupling coefficient is reduced or minimum, which may facilitate homeostasis (health).

In some implementations, transmission of radiant energy through the vascular system overcomes the blood-brain-barrier, inner-blood-retinal barrier, outer-blood-retinal barrier, blood-aqueous-barrier or any combination thereof.

Accordingly, as described herein, in some cases, for example, the laser light (radiant energy) includes a plurality (e.g., 1, 2, 3, 4, 5, 6 or more) of ranges of wavelength such as NIR, MIR, FIR, red and yellow visible lights as well as a plurality (e.g., 1, 2, 3, 4, 5, 6, or more) of different modulations (e.g., in the MHz range) to produce optical pulses that are associated with the variations in sequence, time, duration or fluence or any combination of these and result in patterns, such as those of geometric structures, with fractal shape and/or characteristics. In some implementations, the fractal is a non-linear tridimensional deterministic fractal. This fractal, may possibly be a strange fractal type potentially like the Lorenz's fractal.

Without subscribing to any particular theory, in higher biological systems, water molecules can potentially form a universal attractor. This theory was proposed by Feynman, who described the mechanism of paradoxical attraction in aqueous systems, where negatively charged particles may attract if enough positive charges lie in between. Later, the theory was experimentally confirmed by Norio Ise.

Water may be a nonlinear dynamic system operating at non-equilibrium, with 3 degrees of freedom due to water molecule trajectories, plus 0.5 degrees of freedom linked to time dependence. These characteristics of water are shared with other strange attractors, more specifically with Lorenz's attractors.

The concurrence of the structure of the signal (e.g., the radiant energy of the laser in the ranges proposed and with the proposed features) and/or the proposed regimen (e.g., durations, number of sessions) with the characteristics of water in tissues may potentially allow coupling at different spacetime scales, even for molecules with low coupling coefficient, and results in an efficient transfer of energy within physiological ranges. Thermodynamically, there may be a trajectory of energy subsequently inducing cell tissue homeostasis which improves health.

Likewise, in various implementations, light and/or terminals can be applied symmetrically non-invasively to the skin on or above sites selected potentially based on the fractal geometry of the vascular system as well as the BCEC and/or its sub-set VICC. The latter may exist with the wall of arteries and veins functioning as relative insulators around the electrically conductive medium of blood, the plasma. The vascular system, like the nervous and respiratory systems among others, exhibits fractal geometry. Without subscribing to any particular theory, light energy propagates through the light-sensitive extended annular endovascular structure known as EZ water. Flow in the fluid-filled interstitial spaces (including the submucosal space, dermis, fascia, vascular adventitia possibly with reticular patterns and collagen bundles) may also actively be increased by the longitudinal effect of the change in the hydronium, resulting from the formation of EZ.

Without subscribing to any particular scientific theory, energy may be primarily absorbed by diseased tissues due to redox potential differences, which can reach as much as 1.5 V between tumors and peripheral tissues, as guided by the second law of thermodynamics and the Osanger's reciprocity principle for non-equilibrium systems. According to the first law of photochemistry, also known as the Grotthuss-Draper law, absorbed light can trigger a photochemical reaction. Energy not absorbed may be dissipated as supported by the dissipative extension of Kleiber's law.

The human body can be represented as a complex, electrochemical (semi-conducting) system that comprises a vast array of energy-sensitive materials and machinery, including ion pumps (e.g., chemically driven electron pumping through molecular wires such as D pathways in CcO), molecular motors (e.g., ATP synthetase and Brownian bio-motors (e.g., metallo-colagenase1 and kinesin), switches, transistors, capacitors, liquid crystals (e.g. membranes), and rechargeable electrolytic biobatteries (e.g. hydrophilic interface in cells/tissues). The body also comprises wide metabolic, neural, gene, lipids, protein networks that exhibit dynamically-intricate rhythms arising from nonlinear stochastic biological mechanisms interacting with an oscillating chaotic environment exhibiting quantic and thermal fluctuations.

Targeted CDs can be associated with perturbation of physiological rhythms (PRs). However, reestablishment of PRs or oscillations is complex because their non-linear (chaotic) dynamics originates from the combined influence of noise inherent to biological systems as well as deterministic and non-fully deterministic mechanisms within a chaotic environment.

Again, without subscribing to any particular scientific theory, various implementations described herein may potentially induce coupling of the photic energy supplied with multiple ligands at different time-space scales that follow a dynamic system and intervene in the restitution of physiological rhythms. Proposed action mechanisms may include: an oxygen dependent pathway linked to the electron chain transport in complexes I to IV and a second complementary or supplementary pathway called oxygen independent, comprised of (1) molecular hydrophobic forces such as folding/unfolding of proteins and DNA/RNA self-assembly and (2) hydrophilic interfaces, including the exclusion zone (EZ), which has been shown to separate and store charges, thus acting as a potential energy reservoir. Finally, light energy transport across areas such as blood-brain-barrier, inner-blood-retinal barrier, outer-blood-retinal barrier, and blood-aqueous-barrier in the eye can overcome restrictions on the movement of molecules or ions from inside the blood vessels to avascular spaces in the eye and brains as light energy is transferred without mass.

In some cases, multiple photo-acceptors that act as redox centers may be targeted, including but not limited to: water, carbon dioxide (CO₂), hydrogen bonds, TPR channels, lipids, cofactors, photopigments, metal ions, proteins, enzymes, nucleic acids and other chromophores as well as subcellular structures (e.g. mitochondria/CcO, lysosome, and/or endothelial reticule).

Without subscribing to any particular theory, radiant multifrequency energy, emitted following nonlinear fractional rhythms (e.g., multifractal regimes), can possibly allow increased or even potentially maximize coupling with the different ligands at different scales of time-space rhythms that regulate the physiology of higher biological systems. Energy absorbed may potentially induce non-thermal, photochemical-photophysical effects, that can power and modulate reparative and/or regenerative mechanisms, promoting the reestablishment of homeokinesis/homeostasis, i.e., health.

Again, without subscribing to any particular theory, CDs can potentially arise from the combined action of genes, the environment, and risk-conferring behaviors. While different CDs have their own etiology and pathogenesis, different CDs often induce similar morphological and functional perturbations, including deregulation of programmed cell death. Oxidative stress, altered signaling pathways, impaired bioenergetic and immune systems, deregulated inflammatory response, aging, epigenetic effects, vascular compromise, alteration of microbiota, neurodegenerative changes, dysregulation of stem cells, cellular dedifferentiation/trans-differentiation are also common factors that disturb tissues' dynamic balance, known as homeostasis/homeokinesis.

Accordingly, various methods and systems described herein can potentially use pulsed EM signals (e.g., light) aimed at specific photo-acceptors (e.g., water, CO₂, hydrogen bonds, proteins, TPR channels, lipids, photopigments, metal ions, cofactors, enzymes, and other chromophores), can modulate (e.g., inhibit or stimulate) molecules, subcellular organelles, cell and tissue functions and power reparative and/or regenerative effects that can help reestablish systemic homeokinesis/homeostasis.

In some cases, for example, approaches described herein may potentially substitute and/or complement metabolic energy pathways through both the described oxygen-dependent pathway and novel oxygen-independent action mechanisms. The latter are primarily based on NIR radiant energy absorption and transport by water and CO₂. Water dynamics can absorb, transduce and transport energy along extended biological surfaces including avascular eye tissues (i.e., cornea, lens, aqueous humor, vitreous, and foveal avascular zone). This pathway further integrates the role of crystalline water, known also as the fourth phase of water or exclusion zone (EZ) water. EZ water represents ⅔ of the content of the intracellular confine space, where it is mainly associated with the crowded protein interface and acts as a selective rechargeable electrolytic bio-battery due to its charge separation and release properties. Water represents approximately 70% of the weight of an adult and over 90% of molecules in the human body.

Together or separately, one or both of these two pathways can power and modulate multi-hallmark effects in cancer and other CDs and induce neuroprotective, vasoprotective, baroprotective, immunomodulator, and regenerative effects, locally and remotely, promoting systemic homeostasis/homeokinesis. This is possible at all time-space scales through the coupling and synchronization of biophysical, biochemical, biomechanical, and hydrodynamic oscillators.

Since the biological system operates under non-equilibrium, the energy regularly supplied in the application points recharges the biobattery, contributes to the metabolic redox equilibrium and intervenes as a fundamental cell signaling pathway, inducing homeostasis/homeokinesis. The initial molecular agent responsible for liquid flow and cell signaling effects is the hydronium (H₃O) produced by the abundant release of protons from the surface of the biobattery and the ample amount of water molecules in the vascular circulating media. The hydroxyls (electrons) transfer charge to the tissues for cell work.

A parallel biobattery system can be formed in the intramural submucosae of the blood vessels and other tissues, at the level of the pre-lymphatic reticular anatomic structures. The biobattery favors the increased vascular and interstitial flow, and with it, the elimination of metabolic waste (e.g., prion proteins, beta-amyloids, etc.) and the destruction of circulating metastatic cells caused by the action of the hydronium. Energy is primarily absorbed by diseased tissues due to injury redox potential differences and promotes homeostasis/homeokinesis through the coupling and synchronization of biophysical, biochemical, biomechanical, and hydrodynamic oscillators guided by the second law of thermodynamics and the reciprocity principle described by Osanger for non-equilibrium systems.

Some example attributes of various methods that can be employed are provided below. In some implementations, for example, the total daily dose or daily accumulated dose is applied within a 12 hours time period between 7:00 AM and 7:00 PM. The total daily dose or daily accumulated dose can be distributed in multiple sessions, for example, possibly ranging from 2 to 8 sessions per day. In various implementations, the different sessions can vary in length, for example, from 5 to 25 minutes. In some implementations, the treatment is applied on a daily basis, and may be provided for prolonged periods of time. The methods of treatment and therapy, however, should not be limited to any one or more of these particular possible attributes.

As discussed above, an apparatus may non-invasively provide light to a human or an animal to provide treatment, for example, for tumors, eye disease, neurological diseases or other diseases or medical conditions. In particular, various methods and systems described herein may be applicable to treating cancer (e.g., solid tumors, not including myeloid diseases), diseases of the eye (e.g., glaucoma, age-related macular degeneration (AMD), diabetic retinopathy (DR), retinitis pigmentosa (RP)), diseases of the brain (e.g., Alzheimer's (AD) and Parkinson's disease (PD)), arterial diseases (e.g., peripheral arterial disease (PAD)) or possibly additional diseases, conditions or ailments.

The methods described herein may offer one or more possible, non-limiting advantages. For example, the method, which is non-invasive and employs non-ionizing, non-thermal optical radiation is safe. This safeness may derive in part from the methods, its technical characteristics and/or mechanistic principles. As described above, the method is non-invasive and/or relatively easy to implement. Likewise, the system may be portable and/or easy to operate.

The method and/or system may provide the ability to deliver multi-target, multi-hallmark, monotherapy for CDs with few or no current proven treatment options, including solid tumors, PAD and diseases of the eye and brain. The action mechanism proposed, which can be administered without direct application to optic tissues, can thereby prevent long term accumulated toxicity when treating eye diseases. Other advantages are possible, while achieving none of these advantages is required.

### EXAMPLES

This disclosure provides various examples of devices, systems, and methods. Some such examples include but are not limited to the following examples.

### Part I

1. A method of treating a human or animal having a body using pulses of light, said method comprising:
   non-invasively applying modulated light to a plurality of anatomical locations on said body simultaneously;
   wherein said plurality of anatomical locations includes at least two of the following:
      (a) a location on the skin proximal the left carotid artery and a location on the skin proximal the right carotid artery,
      (b) a location on the skin proximal the left axillary artery and a location on the skin proximal the right axillary artery,
      (c) a location on the skin proximal the left femoral artery and a location on the skin proximal the right femoral artery,
      (d) a location on the skin proximal the left popliteal artery and a location on the skin proximal the right popliteal artery,
      (e) a location on the skin proximal the left tibial artery and a location on the skin proximal the right tibial artery,
      (f) a location on the skin proximal the left dorsalis pedis artery and a location on the skin proximal the right dorsalis pedis artery,
      (g) a location on the skin proximal the left radial artery and a location on the skin proximal the right radial artery,
      (i) a location on the skin proximal the left iliac artery and a location on the skin proximal the right iliac artery, or
      (j) a location on the skin proximal the left posterior tibial artery and a location on the skin proximal the right posterior tibial artery.
2. The method of any of the claims above, wherein said arteries include at least four of the following:
   (a) a location on the skin proximal the left carotid artery and a location on the skin proximal the right carotid artery,
   (b) a location on the skin proximal the left axillary artery and a location on the skin proximal the right axillary artery,
   (c) a location on the skin proximal the left femoral artery and a location on the skin proximal the right femoral artery,
   (d) a location on the skin proximal the left popliteal artery and a location on the skin proximal the right popliteal artery,
   (e) a location on the skin proximal the left anterior tibial artery and a location on the skin proximal the right anterior tibial artery,
   (f) a location on the skin proximal the left dorsalis pedis artery and a location on the skin proximal the right dorsalis pedis artery,
   (g) a location on the skin proximal the left radial artery and a location on the skin proximal the right radial artery,
   (i) a location on the skin proximal the left iliac artery and a location on the skin proximal the right iliac artery, or
   (j) a location on the skin proximal the left posterior tibial artery and a location on the skin proximal the right posterior tibial artery.
3. The method of any of the claims above, wherein said arteries include at least six of the following:
   (a) a location on the skin proximal the left carotid artery and a location on the skin proximal the right carotid artery,
   (b) a location on the skin proximal the left axillary artery and a location on the skin proximal the right axillary artery,
   (c) a location on the skin proximal the left femoral artery and a location on the skin proximal the right femoral artery,
   (d) a location on the skin proximal the left popliteal artery and a location on the skin proximal the right popliteal artery,
   (e) a location on the skin proximal the left anterior tibial artery and a location on the skin proximal the right anterior tibial artery,
   (f) a location on the skin proximal the left dorsalis pedis artery and a location on the skin proximal the right dorsalis pedis artery,
   (g) a location on the skin proximal the left radial artery and a location on the skin proximal the right radial artery,
   (i) a location on the skin proximal the left iliac artery and a location on the skin proximal the right iliac artery, or
   (j) a location on the skin proximal the left posterior tibial artery and a location on the skin proximal the right posterior tibial artery.
4. The method of any of the examples above, wherein light is modulated to produce a plurality of optical pulses having a repetition rate in a range from 1 to 15 MHz.
5. The method of any of the examples above, wherein light is modulated to produce a plurality of optical pulses having a repetition rate in the MegaHertz range.

### Part II

1. A method of treating a human or animal having a body using pulses of light, said method comprising:
   non-invasively applying a plurality of sequences of optical pulses comprising at least a first plurality of pulses of a first wavelength at a first repetition rate to a plurality of anatomical locations on said body as part of a plurality of sessions on one or more days,
   wherein one or more parameters of the plurality of sequences of optical pulses are fractals and/or exhibit properties of fractals.
2. The method of Example 1, wherein after applying the first plurality of pulses of the first wavelength at the first repetition rate, subsequently applying a second plurality of pulses of a second different wavelength at a second repetition rate.
3. The method of any of the examples above, wherein the duration of the application of optical pulses in a given sequence contribute to the one or more parameters being a fractal.
4. The method of any of the examples above, wherein the duration of the application of optical pulses of different wavelengths in a given sequence contribute to the one or more parameters being a fractal.
5. The method of any of the examples above, wherein the duration of the application of optical pulses in a plurality of sequences contribute to the one or more parameters being a fractal.
6. The method of any of the examples above, wherein the duration of the application of optical pulses of different wavelengths in a plurality of sequences contribute to the one or more parameters being a fractal.
7. The method of any of the examples above, wherein the duration of the application of optical pulses in a given session contribute to the one or more parameters being a fractal.
8. The method of any of the examples above, wherein the duration of the application of optical pulses of different wavelengths in a given session contribute to the one or more parameters being a fractal.
9. The method of any of the examples above, wherein the duration of the application of optical pulses over a plurality of sessions contribute to the one or more parameters being a fractal.
10. The method of any of the examples above, wherein the duration of the application of optical pulses of different wavelengths over a plurality of sessions contribute to the one or more parameters being a fractal.
11. The method of any of the examples above, wherein the duration of the application of optical pulses over a plurality of days contribute to the one or more parameters being a fractal.
12. The method of any of the examples above, wherein the duration of the application of optical pulses of different wavelengths over a plurality of days contribute to the one or more parameters being a fractal.
13. The method of any of the examples above, wherein the order of the application of different wavelengths of light in a given sequence or in multiple sequences contribute to the one or more parameters being a fractal.
14. The method of any of the examples above, wherein the order of the application of different wavelengths of light in a given session or in multiple sessions contribute to the one or more parameters being a fractal.
15. The method of any of the examples above, wherein the order of the application of different optical signals from different light sources in a given sequence or in multiple sequences contribute to the one or more parameters being a fractal.
16. The method of any of the examples above, wherein the order of the application of different optical signals from different light sources in a given session or in multiple sessions contribute to the one or more parameters being a fractal.
17. The method of any of the examples above, wherein the number of sessions on a given day contribute to the one or more parameters being a fractal.
18. The method of any of the examples above, wherein the number of sessions over a plurality of days contribute to the parameters being a fractal.
19. The method of any of the examples above, wherein a plurality of sequences exhibit properties of fractals or contribute to the properties of fractals exhibited.
20. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to the one or more parameters being a fractal.
21. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to the parameters being a fractal.
22. The method of any of the examples above, wherein the duration of the application of optical pulses in a given sequence exhibit fractal properties or contribute to the fractal properties exhibited.
23. The method of any of the examples above, wherein the duration of the application of optical pulses of different wavelengths in a given sequence exhibit fractal properties or contribute to the fractal properties exhibited.
24. The method of any of the examples above, wherein the duration of the application of optical pulses in a plurality of sequences exhibit fractal properties or contribute to the fractal properties exhibited.
25. The method of any of the examples above, wherein the duration of the application of optical pulses of different wavelengths in a plurality of sequences exhibit fractal properties or contribute to the fractal properties exhibited.
26. The method of any of the examples above, wherein the duration of the application of optical pulses in a given session exhibit fractal properties or contribute to the fractal properties exhibited.
27. The method of any of the examples above, wherein the duration of the application of optical pulses of different wavelengths in a given session exhibit fractal properties or contribute to the fractal properties exhibited.
28. The method of any of the examples above, wherein the duration of the application of optical pulses over a plurality of sessions exhibit fractal properties or contribute to the fractal properties exhibited.
29. The method of any of the examples above, wherein the duration of the application of optical pulses of different wavelengths over a plurality of sessions exhibit fractal properties or contribute to the fractal properties exhibited.
30. The method of any of the examples above, wherein the duration of the application of optical pulses over a plurality of days exhibit fractal properties or contribute to the fractal properties exhibited.
31. The method of any of the examples above, wherein the duration of the application of optical pulses of different wavelengths over a plurality of days exhibit fractal properties or contribute to the fractal properties exhibited.
32. The method of any of the examples above, wherein the order of the application of different wavelengths of light in a given sequence or in multiple sequences exhibit fractal properties or contribute to the fractal properties exhibited.
33. The method of any of the examples above, wherein the order of the application of different wavelengths of light in a given session or in multiple sessions exhibit fractal properties or contribute to the fractal properties exhibited.
34. The method of any of the examples above, wherein the order of the application of different optical signals from different light sources in a given sequence or in multiple sequences exhibit fractal properties or contribute to the fractal properties exhibited.
35. The method of any of the examples above, wherein the order of the application of different optical signals from different light sources in a given session or in multiple sessions exhibit fractal properties or contribute to the fractal properties exhibited.
36. The method of any of the examples above, wherein the number of sessions on a given day exhibit fractal properties or contribute to the fractal properties exhibited.
37. The method of any of the examples above, wherein the number of sessions over a plurality of days exhibit fractal properties or contribute to the fractal properties exhibited.
38. The method of any of the examples above, wherein a plurality of sequences exhibit fractal properties or contribute to the fractal properties exhibited.
39. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit fractal properties or contribute to the fractal properties exhibited.
40. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit one or more properties of a multifractal system or contribute to the properties of fractals exhibited.
41. The method of any of the examples above, wherein said fractal property or said fractal exhibits a fractal property matched to or within a threshold difference of a fractal property of a biological signal, system, structure or process.
42. The method of Example 41, wherein said fractal property that is matched to or within a threshold difference of a fractal property of a biological signal, system, structure or process comprises fractal dimension.
43. The method of Examples 41 or 42, wherein said biological signal, system, structure or process comprises a biological signal, system, structure or process that is affected by or contributes to a disease, medical condition or ailment being treated by said method.
44. The method of any of the examples above, wherein said fractal exhibits fractal characteristic that is within a threshold difference from a fractal characteristic of a biological signal, system, structure or process.
45. The method of Example 44, wherein said fractal characteristic that is within a threshold difference from the fractal characteristic of a biological signal, system, structure or process comprises fractal dimension.
46. The method of Examples 44 or 45, wherein said biological signal, system, structure or process comprises a biological signal, system, structure or process that is affected by or contributes to a disease, medical condition or ailment being treated by said method.
47. The method of any of Examples 41-46, wherein said biological signal, system, structure, or process derives from or comprises water in said body.
48. The method of any of the examples above, further comprising using water as a universal attractor.
49. The method of any of any of the examples above, further comprising using water molecules as an extended rechargeable electrolytic biobattery.
50. The method of any of any of the examples above, wherein transmission of radiant energy through the vascular system overcomes the blood-brain-barrier, inner-blood-retinal barrier, outer-blood-retinal barrier, blood-aqueous-barrier of the eye or any combination thereof.
51. The method of any of any of the examples above, wherein transmission of radiant energy through the vascular system induces coupling of photic energies supplied with multiple ligands at different time-space scales that follow a dynamic system and intervene in the restitution of physiological rhythms.
52. The method of any of the examples above, wherein said plurality of sequences of optical pulses and/or one of said sequences of optical pulses has a variable temporal fractal dimension.
53. The method of any of the examples above, wherein said plurality of sequences of optical pulses and/or one of said sequences of optical pulses comprises a non-linear, fractional signal.
54. The method of any of the examples above, wherein said fractal dimension comprises at least one non-linear, fractional signal.
55. The method of any of the examples above, wherein said plurality of sequences of optical pulses provides a daily fractioned dose and accumulated fractioned dose that are based on a multifractal system.

### Part III

1. A method of treating a human or animal having a body using pulses of light, said method comprising:
   non-invasively applying a first sequence of optical pulses comprising at least a first plurality of pulses of a first wavelength at a first repetition rate, and subsequently a second plurality of pulses of a second different wavelength at a second repetition rate to a plurality of anatomical locations on said body as part of said first sequence for a first session on a first day, and
   wherein said first and second repetition rates are in the MegaHertz range.
2. The method of any of the examples above, wherein said first and second repetition rates are in a range from 1 to 15 MHz.
3. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit one or more fractal properties.
4. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit one or more properties of a multifractal system.
5. The apparatus of any of the examples above, wherein said optical pulses are applied to skin proximal to arteries.
6. The method of any of the claims above, wherein said optical pulses are applied to skin within 30 mm of a location directly above said arteries.
7. The method of any of the claims above, wherein said optical pulses are applied to skin proximal counterpart left and right arteries.

### Part IV

1. An apparatus for treating a human or animal having a body using pulses of light, said apparatus comprising:
   a plurality of light sources, said plurality of light sources comprising at least first and second light sources that output light having different wavelengths than each other,
   a plurality of optical fibers having proximal and distal ends, said optical fibers configured such that respective proximal ends of respective optical fibers receive light from said respective ones of said plurality of light sources, said distal ends configured to be positioned to provide light from said first light source to a plurality of locations on the body proximal to arteries at a first time and to provide light from said second light source to a plurality of locations on the body proximal to said arteries at a second time, and
   electronics configured to execute an illumination regimen comprising:
      applying optical pulses of a first wavelength and a first repetition rate to a plurality of anatomical locations on said body for a first temporal duration;
      after applying said optical pulses of said first wavelength, applying optical pulses of a second wavelength and a second repetition rate to said plurality of anatomical locations on said body for a second temporal duration,
      wherein said first and second wavelengths are in the near infrared, mid infrared, far infrared or visible wavelengths or any combination thereof.
2. The apparatus of Example 1, wherein said electronics is configured to provide and execute said regimen.
3. The apparatus of any of the examples above, wherein said electronics are configured to modulate light output by said light sources so as to provide optical pulses.
4. The apparatus of any of the examples above, wherein said electronics are configured to modulate said light output by said light sources so as to provide optical pulses wherein first and second repetition rates are in the MegaHertz range.
5. The apparatus of any of the examples above, wherein said electronics are configured to modulate said light output by said light sources so as to provide optical pulses wherein first and second repetition rates are in a range from 1 to 15 MHz.
6. The apparatus of any of the examples above, wherein in said illumination regimen the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to one or more fractal properties.
7. The apparatus of any of the examples above, wherein in said illumination regimen the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to properties of a multifractal system.
8. The apparatus of any of the examples above, wherein said optical fibers are configured to applied said light pulses to the skin proximal to arteries.
9. The apparatus of any of the examples above, wherein said optical fibers are configured to applied said light pulses to the skin within 30 mm of a location directly above said arteries.
10. The apparatus of any of the examples above, wherein said optical fibers are configured to applied said light pulses to skin proximal counterpart left and right arteries.

### Part V

1. A method of treating a body with light pulses, said method comprising simultaneous, symmetric, non-invasive application of multiple nodes or terminals to the skin directly above or in close proximity the following arteries: 1) carotid, 2) axillary 3) femoral, 4) popliteal, 5) tibial and 6) dorsalis pedis, without excluding the symmetric application in other arteries of the human anatomy.
2. This method further Example 1 comprising one or more near infrared wavelength, sole or combined with one or more of the other wavelengths included below:
(a) near infrared: 780-810 nm wavelength output pulsed at 9.5-10 MHz,
(b) near infrared: 904-945 nm wavelength output pulsed at 3-3.5 MHz,
(c) mid infrared: 1200-1,550 nm wavelength output pulsed at 6-6.5 MHz,
(d) far infrared: 2,900-3,200 nm wavelength output pulsed at 7-7.5 MHz,
(e) red: 630-700 nm wavelength output pulsed at 4-4.5 MHz, or
(f) yellow: 570-600 nm wavelength output pulsed at 5-5.5 M.
3. The method of any of the examples above including using one or more laser diodes to produce the light, wherein a peak power of each diode is from 25-100 mW, the pulse duration is 200 ns, and the laser diode provides a duty cycle of from 40-50%.
4. The method of any of the examples above, further comprising an on/off signal sequence that follows a variable temporal fractal dimension. Such fractal dimension can also be understood as a non-linear, fractional signal.
5. The method of any of the examples above, wherein a daily fractioned dose and an accumulated fractioned dose are based on a multifractal system.
6. The method of any of the examples above, wherein a total daily dose or daily accumulated dose is applied within a 12 hour time period between 07:00 hours and 19:00 hours.
7. The method of any of the examples above, wherein the daily dose or daily accumulated dose is distributed in multiple sessions, ranging from 2 to 8 sessions per day.
8. The method of any of the examples above, wherein each session varies in length from 5 to 25 minutes.
9. The method of any of the examples above, wherein treatment is applied on a daily basis, for prolonged periods of time.

### Part VI

1. A method of treating a human or animal having a body using pulses of light, said method comprising:
   non-invasively applying a first sequence of optical pulses comprising at least a first plurality of pulses of a first wavelength at a first repetition rate, and subsequently a second plurality of pulses of a second different wavelength at a second repetition rate to a plurality of anatomical locations on said body as part of said first sequence for a first session on a first day,
   wherein said plurality of anatomical locations is exterior of the body and proximal to arteries.
2. The method of Example 1, wherein said optical pulses of said second wavelength have a different repetition rate as the optical pulses of the first wavelength.
3. The method of Example 1 or 2, wherein said optical pulses of said second wavelength are applied for a different duration than the optical pulses of the first wavelength.
4. The method of Example 1 or 2, wherein said optical pulses of said second wavelength are applied for a same duration than the optical pulses of the first wavelength.
5. The method of any of the examples above, wherein said first sequence further comprises applying a plurality of optical pulses of a third wavelength different from the first and second wavelengths as part of said first sequence in said first session on said first day.
6. The method of Example 5, wherein said optical pulses of said third wavelength have a different repetition rate as the optical pulses of the second wavelength.
7. The method of Example 5 or 6, wherein said optical pulses of said third wavelength are applied for a different duration than the optical pulses of the second wavelength.
8. The method of Example 5 or 6, wherein said optical pulses of said third wavelength are applied for a same duration than the optical pulses of the second wavelength.
9. The method of any of the examples above, wherein said first sequence further comprises applying a plurality of pulses of a fourth wavelength different from the first, second, and third wavelengths.
10. The method of Example 9, wherein said optical pulses of said fourth wavelength have a different repetition rate as the optical pulses of the third wavelength.
11. The method of Example 9 or 10, wherein said optical pulses of said fourth wavelength are applied for a different duration than the optical pulses of the third wavelength.
12. The method of Example 9 or 10, wherein said optical pulses of said fourth wavelength are applied for a same duration than the optical pulses of the third wavelength.
13. The method of any of the examples above, wherein said first sequence further comprises applying a plurality of pulses of a fifth wavelength different from the first, second, third, and fourth wavelengths.
14. The method of Example 13, wherein said optical pulses of said fifth wavelength have a different repetition rate as the optical pulses of the fourth wavelength.
15. The method of Example 13 or 14, wherein said optical pulses of said fifth wavelength are applied for a different duration than the optical pulses of the fourth wavelength.
16. The method of Example 13 or 14, wherein said optical pulses of said fifth wavelength are applied for a same duration than the optical pulses of the fourth wavelength.
17. The method of any of the examples above, wherein said first sequence further comprises applying a plurality of pulses of a sixth wavelength different from the first, second, third, fourth, and fifth wavelengths.
18. The method of Example 17, wherein said optical pulses of said sixth wavelength have a different repetition rate as the optical pulses of the fifth wavelength.
19. The method of Example 17 or 18, wherein said optical pulses of said sixth wavelength are applied for a different duration than the optical pulses of the fifth wavelength.
20. The method of Example 17 or 18, wherein said optical pulses of said sixth wavelength are applied for a same duration than the optical pulses of the fifth wavelength.
21. The method of any of the examples above, wherein the first sequence further comprises repeating application of first optical pulses having a first central wavelength after second optical pulses of a second different central wavelength have been applied after optical pulses of the said first central wavelength have been previously used in said first sequence on the same day.
22. The method of Example 21, wherein said first optical pulses of said first central wavelength are applied for a different duration than the optical pulses of the same central wavelength that have been previously used in said first sequence on said same day.
23. The method of Example 21, wherein said first optical pulses of said first central wavelength are applied for the same duration as the optical pulses of the same central wavelength that have been previously used in said first sequence on said same day.
24. The method of any of Examples 1-20, wherein said first sequence further comprises repeating application of first and second optical pulses having two different central wavelengths, respectively, after optical pulses having a different central wavelength have been applied after optical pulses of the same two central wavelengths have been previously used in said first sequence on said same day.
25. The method of Example 24, wherein said first optical pulses having at least one of said two central wavelengths are applied for a different duration than respective optical pulses of the same central wavelength that have been previously used in said first sequence on said same day.
26. The method of Example 24, wherein said first optical pulses of at least one of said two central wavelengths are applied for a same duration as the optical pulses of the same central wavelength that have been previously used in said first sequence on said same day.
27. The method of Example 24, wherein said first and second optical pulses of both said two central wavelengths, respectively, are applied for a different duration than the optical pulses of the same respective central wavelengths that have been previously used in said first sequence on said same day.
28. The method of Example 24, wherein said first and second optical pulses of both said two central wavelengths, respectively, are applied for a same duration as the optical pulses of the same respective central wavelengths that have been previously used in said first sequence on said same day.
29. The method of any of Examples 1-28, wherein in a second session after said first session on the first day, the duration of application of at least one of the wavelengths in said first sequence changes.
30. The method of any of Examples 1-28, wherein in a second session after the first session of the first day, respective durations of application of a plurality of the wavelengths in said first sequence changes.
31. The method of any of the examples above, wherein in a third session after the second session on the first day, the duration of application of at least one of the wavelengths in said second sequence changes with respect to that of the second session.
32. The method of any of the examples above, wherein in a third session after the second session on the first day, respective durations of application of a plurality of the wavelengths in the second sequence changes with respect to the second session.
33. The method of any of the examples above, wherein in a first session of a second day, the duration of application of at least one of the wavelengths in the first sequence changes with respect to the first session of the first day.
34. The method of any of the examples above, wherein in a first session of the second day, respective durations of application of a plurality of the wavelengths in the first sequence changes with respect to the first session of the first day.
35. The method of any of the examples above, wherein in a second session of the second day, the duration of application of at least one of the wavelengths in the first sequence changes with respect to the first session of the first day and with respect to the first session of the second day.
36. The method of any of the examples above, wherein in a second session of the second day, respective durations of application of a plurality of the wavelengths in the first sequence changes with respect to the first session of the first day and with respect to the first session of the second day.
37. The method of any of the examples above, wherein a plurality of optical pulses are applied on a second day and wherein the aggregate duration of the pulses of light applied on a second day has different duration than the aggregate duration of application of the plurality of optical pulses on the first day.
38. The method of any of the examples above, wherein on said first day a plurality of pulses of light are applied in a plurality of sessions and wherein on a second day a plurality of pulses of light are applied in a plurality of sessions, wherein the number of sessions on the second day is different than the number of sessions on the first day.
39. The method of Example 38, wherein on a third day a plurality of pulses of light are applied in a plurality of sessions, wherein the number of sessions on the third day is different than the number of sessions on the second day.
40. The method of any of the examples above, wherein on a second day a plurality of pulses of light having different wavelengths are applied in a second sequence that is different than said first sequence used on said first day.
41. The method of Example 40, wherein said second sequence has a different order of application of light of different wavelengths than said first sequence.
42. The method of Example 40 or 41, wherein said second sequence has at least one wavelength added that is not in said first sequence.
43. The method of any of Examples 40-42, wherein said second sequence has at least one wavelength removed from said first sequence.
44. The method of any of Examples 40-43, wherein on a third day a plurality of pulses of light having different wavelengths are applied in a third sequence that is different than said second sequence used on said second day.
45. The method of Example 44, wherein said third sequence has a different order of application of light of different wavelengths than said second sequence.
46. The method of Example 44 or 45, wherein said third sequence has at least one wavelength added that is not in said second sequence.
47. The method of any of Examples 44-46, wherein said third sequence has at least one wavelength removed from said second sequence.
48. The method of any of the examples above, wherein in a second session on said first day a plurality of pulses of light having different wavelengths are applied in a second sequence that is different than said first sequence used on said first day.
49. The method of Example 48, wherein said second sequence has a different order of application of light of different wavelengths than said first sequence.
50. The method of Example 48 or 49, wherein said second sequence has at least one wavelength added that is not in said first sequence.
51. The method of any of Examples 48-50, wherein said second sequence has at least one wavelength removed from said first sequence.
52. The method of any of Examples 48-51, wherein in a third session on said first day a plurality of pulses of light having different wavelengths are applied in a third sequence that is different than said second sequence used on said first day.
53. The method of Example 52, wherein said third sequence has a different order of application of light of different wavelengths than said second sequence.
54. The method of Example 52 or 53, wherein said third sequence has at least one wavelength added that is not in said second sequence.
55. The method of any of Example 52-54, wherein said third sequence has at least one wavelength removed from said second sequence.
56. The method of any of the examples above, wherein the duration of the application of optical pulses of different wavelengths in a given sequence exhibit one or more fractal properties.
57. The method of any of the examples above, wherein the duration of the application of optical pulses of different wavelengths over a plurality of sequences exhibit one or more fractal properties.
58. The method of any of the examples above, wherein the duration of the application of optical pulses in a given session exhibit one or more fractal properties.
59. The method of any of the examples above, wherein the duration of the application of optical pulses over a plurality of sessions exhibit one or more fractal properties.
60. The method of any of the examples above, wherein the duration of the application of optical pulses over a plurality of days exhibit one or more fractal properties.
61. The method of any of the examples above, wherein the number of sessions on a given day exhibit one or more fractal properties.
62. The method of any of the examples above, wherein the number of sessions over a plurality days exhibit one or more fractal properties.
63. The method of any of the examples above, wherein a plurality of sequences exhibits one or more fractal properties.
64. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit one or more fractal properties.
65. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof exhibit one or more properties of a multifractal system.
66. The method of any of the examples above, wherein said first and second repetition rates are in the MegaHertz range.
67. The method of any of the examples above, wherein said first and second repetition rates are in a range from 1 to 15 MHz.
68. The method of any of the examples above, wherein said first repetition rate is different from said second repetition rate.
69. The method of any of the examples above, wherein said first and second wavelengths are from two of the following ranges: 780 to 810 nm, from 904 to 945 nm, from 1,200 to 1,550 nm, from 2,900 to 3,200 nm, 630 to 700 nm or from 570 to 600 nm.
70. The method of any of the examples above, wherein said first and second repetition rate are from two of the following ranges: 3 to 3.5 MHz, 4 to 4.5 MHz, 5 to 5.5 MHz, 6 to 6.5 MHz, 7 to 7.5 MHz, or 9.5 to 10 MHz.
71. The method of any of the examples above, wherein said sequence further comprises applying optical pulses from at least one other light source having a different wavelength after applying said optical pulses of said first wavelength.
72. The method of any of the examples above, further comprising applying optical pulses of a third wavelength and a third repetition rate to said plurality of anatomical locations on said body for a third temporal duration after applying said optical pulses of said first and second wavelengths.
73. The method of Example 72, further comprising applying optical pulses of a fourth wavelength and a fourth repetition rate to said plurality of anatomical locations on said body for a third temporal duration after applying said optical pulses of said first, second and third wavelengths.
74. The method of Example 73, further comprising applying optical pulses of a fifth wavelength and a fifth repetition rate to said plurality of anatomical locations on said body for a fifth temporal duration after applying said optical pulses of said first, second, third, and fourth wavelengths.
75. The method of Example 74, further comprising applying optical pulses of a sixth wavelength and a sixth repetition rate to said plurality of anatomical locations on said body for a sixth temporal duration after applying said optical pulses of said first, second, third, fourth, and fifth wavelengths.
76. The method of any of the examples above, further comprising applying optical pulses of said first wavelength and said first repetition rate to said plurality of anatomical locations on said body after applying said optical pulses of said first and second wavelengths.
77. The method of any of the examples above, further comprising applying optical pulses of said second wavelength and said second repetition rate to said plurality of anatomical locations on said body after applying said optical pulses of said another wavelength besides said second wavelengths.
78. The method of any of the examples above, further comprising applying optical pulses of a third wavelength and a third repetition rate to said plurality of anatomical locations on said body for after applying said optical pulses of said another wavelength besides said second wavelengths.
79. The method of any of the examples above, wherein said light pulses are applied to the skin proximal to arteries.
80. The method of any of the examples above, wherein said light pulses are applied to the skin directly above said arteries.
81. The method of any of the examples above, wherein said light pulses are applied to the skin within 50 mm of a location directly above said arteries.
82. The method of any of the examples above, wherein said light pulses are applied to the skin within 30 mm of a location directly above said arteries.
83. The method of any of the examples above, wherein said light pulses are applied to the skin within 15 mm of a location directly above said arteries.
84. The method of any of the examples above, wherein said light pulses are applied to skin proximal counterpart left and right arteries.
85. The method of any of the examples above, wherein said arteries include at least two of the following:
   (a) a location on the skin proximal the left carotid artery and a location on the skin proximal the right carotid artery,
   (b) a location on the skin proximal the left axillary artery and a location on the skin proximal the right axillary artery,
   (c) a location on the skin proximal the left femoral artery and a location on the skin proximal the right femoral artery,
   (d) a location on the skin proximal the left popliteal artery and a location on the skin proximal the right popliteal artery,
   (e) a location on the skin proximal the left anterior tibial artery and a location on the skin proximal the right anterior tibial artery,
   (f) a location on the skin proximal the left dorsalis pedis artery and a location on the skin proximal the right dorsalis pedis artery,
   (g) a location on the skin proximal the left radial artery and a location on the skin proximal the right radial artery,
   (i) a location on the skin proximal the left iliac artery and a location on the skin proximal the right iliac artery, or
   (j) a location on the skin proximal the left posterior tibial artery and a location on the skin proximal the right posterior tibial artery.
86. The method of any of the examples above, wherein said arteries include at least four of the following:
   (a) a location on the skin proximal the left carotid artery and a location on the skin proximal the right carotid artery,
   (b) a location on the skin proximal the left axillary artery and a location on the skin proximal the right axillary artery,
   (c) a location on the skin proximal the left femoral artery and a location on the skin proximal the right femoral artery,
   (d) a location on the skin proximal the left popliteal artery and a location on the skin proximal the right popliteal artery,
   (e) a location on the skin proximal the left anterior tibial artery and a location on the skin proximal the right anterior tibial artery,
   (f) a location on the skin proximal the left dorsalis pedis artery and a location on the skin proximal the right dorsalis pedis artery,
   (g) a location on the skin proximal the left radial artery and a location on the skin proximal the right radial artery,
   (i) a location on the skin proximal the left iliac artery and a location on the skin proximal the right iliac artery, or
   (j) a location on the skin proximal the left posterior tibial artery and a location on the skin proximal the right posterior tibial artery.
87. The method of any of the examples above, wherein said arteries include at least six of the following:
   (a) a location on the skin proximal the left carotid artery and a location on the skin proximal the right carotid artery,
   (b) a location on the skin proximal the left axillary artery and a location on the skin proximal the right axillary artery,
   (c) a location on the skin proximal the left femoral artery and a location on the skin proximal the right femoral artery,
   (d) a location on the skin proximal the left popliteal artery and a location on the skin proximal the right popliteal artery,
   (e) a location on the skin proximal the left anterior tibial artery and a location on the skin proximal the right anterior tibial artery,
   (f) a location on the skin proximal the left dorsalis pedis artery and a location on the skin proximal the right dorsalis pedis artery,
   (g) a location on the skin proximal the left radial artery and a location on the skin proximal the right radial artery,
   (i) a location on the skin proximal the left iliac artery and a location on the skin proximal the right iliac artery, or
   (j) a location on the skin proximal the left posterior tibial artery and a location on the skin proximal the right posterior tibial artery.
88. The method of any of the examples above, wherein the duration of the application of optical pulses of different wavelengths in a given sequence contribute to one or more fractal properties.
89. The method of any of the examples above, wherein the duration of the application of optical pulses of different wavelengths over a plurality of sequences contribute to one or more fractal properties.
90. The method of any of the examples above, wherein the duration of the application of optical pulses in a given session contribute to one or more fractal properties.
91. The method of any of the examples above, wherein the duration of the application of optical pulses over a plurality of sessions contribute to one or more fractal properties.
92. The method of any of the examples above, wherein the duration of the application of optical pulses over a plurality of days contribute to one or more fractal properties.
93. The method of any of the examples above, wherein the number of sessions on a given day contribute to one or more fractal properties.
94. The method of any of the examples above, wherein the number of sessions over a plurality days contribute to one or more fractal properties.
95. The method of any of the examples above, wherein a plurality of sequences contribute to one or more fractal properties.
96. The method of any of the examples above, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to one or more fractal properties.
97. The method of any of the examples above, wherein said fractal property or said fractal exhibits a fractal property matched to or within a threshold difference of a fractal property of a biological signal, system, structure or process.
98. The method of Example 97, wherein said fractal property that is matched to or within a threshold difference of a fractal property of a biological signal, system, structure or process comprises fractal dimension.
99. The method of Example 97 or 98, wherein said biological signal, system, structure or process comprises a biological signal, system, structure or process that is affected by or contributes to a disease, medical condition or ailment being treated by said method.
100. The method of any of the examples above, wherein said fractal exhibits a fractal characteristic that is within a threshold difference from a fractal characteristic of a biological signal, system, structure or process.
101. The method of Example 100, wherein said fractal characteristic that is within a threshold difference from the fractal characteristic of a biological signal, system, structure or process comprises fractal dimension.
102. The method of Example 100 or 101, wherein said biological signal, system, structure or process comprises a biological signal, system, structure or process that is affected by or contributes to a disease, medical condition or ailment being treated by said method.
103. The method of any of Examples 97-99 or 100-102, wherein said biological signal, system, structure, or process derives from or comprises water in said body.
104. The method of any of the examples above, further comprising using water as a universal attractor.
105. The method of any of any of the examples above, further comprising using water molecules as an extended rechargeable electrolytic biobattery.
106. The method of any of any of the examples above, wherein transmission of radiant energy through the vascular system overcomes the blood-brain-barrier, and the inner-blood-retinal barrier, the outer-blood-retinal barrier, the blood-aqueous-barrier in the eye or any combination thereof.
107. The method of any of any of the examples above, wherein transmission of radiant energy through the vascular system induces coupling of photic energies supplied with multiple ligands at different time-space scales that follow a dynamic system and intervene in the restitution of physiological rhythms.
108. The method of any of any of the examples above, further comprising non-invasively applying a sequence of optical pulses prior to said first sequence comprising at least a plurality of pulses of a wavelength at a repetition rate different than the first wavelength and first repetition rate for a session prior to said first session on a day prior to said first day.

As discussed herein, wavelength may refer to central and/or peak wavelength. Additionally, unless expressly provided otherwise, the words “first”, “second”, “third”, etc. have been used as adjectives only for the purpose of allowing for distinction between the nouns that they modify from one another, and not for the purpose of describing any particular relationship between those nouns.

Also, as used herein, the term “first”, e.g., applying a first wavelength (e.g., first central wavelength), a first pulse modulation repetition rate, a first signal, a first light component, light from a first light source or a first optical fiber or first optical fiber bundle, a first sequence, a first session, a first day, etc., does not preclude other prior similar actions, step, events, item, element, component or group, etc., for example, application of one or more other wavelengths (e.g., central wavelengths), one or more other pulse modulation repetition rates, one or more other signals, one or more other light components, light from one or more other light sources or optical fibers or optical fiber bundles, one or more other sequences, one or more other sessions, one or more other days, etc., prior. Rather, the term first is used as a matter of convenience for identification of an action, step, event, item, element, component or group, etc. First optical pulses of a first wavelength (e.g., first central wavelength) in a first sequence in a first session on a first day can thus be preceded by prior application of optical pulses of another wavelength and/or another repetition rate in the same or a prior session on the same or prior day.

### Other Considerations

Each of the processes, methods, and algorithms described herein and/or depicted in the attached figures may be embodied in, and fully or partially automated by, code modules executed by one or more physical computing systems, hardware computer processors, application-specific circuitry, and/or electronic hardware configured to execute specific and particular computer instructions. For example, computing systems can include general purpose computers (e.g., servers) programmed with specific computer instructions or special purpose computers, special purpose circuitry, and so forth. A code module may be compiled and linked into an executable program, installed in a dynamic link library, or may be written in an interpreted programming language. In some implementations, particular operations and methods may be performed by circuitry that is specific to a given function.

Further, certain implementations of the functionality of the present disclosure are sufficiently mathematically, computationally, or technically complex that application-specific hardware or one or more physical computing devices (utilizing appropriate specialized executable instructions) may be necessary to perform the functionality, for example, due to the volume or complexity of the calculations involved or to provide results substantially in real-time.

Code modules or any type of data may be stored on any type of non-transitory computer-readable medium, such as physical computer storage including hard drives, solid state memory, random access memory (RAM), read only memory (ROM), optical disc, volatile or non-volatile storage, combinations of the same and/or the like. The methods and modules (or data) may also be transmitted as generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). The results of the disclosed processes or process steps may be stored, persistently or otherwise, in any type of non-transitory, tangible computer storage or may be communicated via a computer-readable transmission medium.

Any processes, blocks, states, steps, or functionalities in flow diagrams described herein and/or depicted in the attached figures should be understood as potentially representing code modules, segments, or portions of code which include one or more executable instructions for implementing specific functions (e.g., logical or arithmetical) or steps in the process. The various processes, blocks, states, steps, or functionalities can be combined, rearranged, added to, deleted from, modified, or otherwise changed from the illustrative examples provided herein. In some embodiments, additional or different computing systems or code modules may perform some or all of the functionalities described herein. The methods and processes described herein are also not limited to any particular sequence, and the blocks, steps, or states relating thereto can be performed in other sequences that are appropriate, for example, in serial, in parallel, or in some other manner. Tasks or events may be added to or removed from the disclosed example embodiments. Moreover, the separation of various system components in the implementations described herein is for illustrative purposes and should not be understood as requiring such separation in all implementations. It should be understood that the described program components, methods, and systems can generally be integrated together in a single computer product or packaged into multiple computer products. Many implementation variations are possible.

The processes, methods, and systems may be implemented in a network (or distributed) computing environment. Network environments include enterprise-wide computer networks, intranets, local area networks (LAN), wide area networks (WAN), personal area networks (PAN), cloud computing networks, crowd-sourced computing networks, the Internet, and the World Wide Web. The network may be a wired or a wireless network or any other type of communication network.

The systems and methods of the disclosure each have several innovative aspects, no single one of which is solely responsible or required for the desirable attributes disclosed herein. The various features and processes described above may be used independently of one another, or may be combined in various ways. All possible combinations and subcombinations are intended to fall within the scope of this disclosure. Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. No single feature or group of features is necessary or indispensable to each and every embodiment.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted can be incorporated in the example methods and processes that are schematically illustrated. For example, one or more additional operations can be performed before, after, simultaneously, or between any of the illustrated operations. Additionally, the operations may be rearranged or reordered in other implementations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

What is claimed is:

1. A method of treating a human or animal having a body using pulses of light, said method comprising:
non-invasively applying a first sequence of optical pulses comprising at least a first plurality of pulses of a first wavelength at a first repetition rate, and subsequently a second plurality of pulses of a second different wavelength at a second repetition rate to a plurality of anatomical locations on said body as part of said first sequence for a first session on a first day,
wherein said plurality of anatomical locations is exterior of the body and proximal to arteries.

2. The method of claim 1, wherein said optical pulses of said second wavelength have a different repetition rate as the optical pulses of the first wavelength.

3. The method of claim 1, wherein said optical pulses of said second wavelength are applied for a different duration than the optical pulses of the first wavelength.

4. The method of claim 1, wherein said first sequence further comprises applying a plurality of optical pulses of a third wavelength different from the first and second wavelengths as part of said first sequence in said first session on said first day.

5. The method of claim 4, wherein said optical pulses of said third wavelength have a different repetition rate as the optical pulses of the second wavelength.

6. The method of claim 4, wherein said optical pulses of said third wavelength are applied for a different duration than the optical pulses of the second wavelength.

7. The method of claim 1, wherein said first and second repetition rates are in a range from 1 to 15 MHz.

8. The method of claim 1, wherein said first and second wavelengths are from two of the following ranges: 780 to 810 nm, from 904 to 945 nm, from 1,200 to 1,550 nm, from 2,900 to 3,200 nm, 630 to 700 nm or from 570 to 600 nm.

9. The method of claim 1, wherein said first and second repetition rate are from two of the following ranges: 3 to 3.5 MHz, 4 to 4.5 MHz, 5 to 5.5 MHz, 6 to 6.5 MHz, 7 to 7.5 MHz, or 9.5 to 10 MHz.

10. The method of claim 1, wherein said light pulses are applied to the skin proximal to arteries.

11. The method of claim 1, wherein said light pulses are applied to the skin within 50 mm of a location directly above said arteries.

12. The method of claim 1, wherein said light pulses are applied to skin proximal counterpart left and right arteries.

13. The method of claim 1, wherein said arteries include at least two of the following:
(a) a location on the skin proximal the left carotid artery and a location on the skin proximal the right carotid artery,
(b) a location on the skin proximal the left axillary artery and a location on the skin proximal the right axillary artery,
(c) a location on the skin proximal the left femoral artery and a location on the skin proximal the right femoral artery,
(d) a location on the skin proximal the left popliteal artery and a location on the skin proximal the right popliteal artery,
(e) a location on the skin proximal the left anterior tibial artery and a location on the skin proximal the right anterior tibial artery,
(f) a location on the skin proximal the left dorsalis pedis artery and a location on the skin proximal the right dorsalis pedis artery,
(g) a location on the skin proximal the left radial artery and a location on the skin proximal the right radial artery,
(i) a location on the skin proximal the left iliac artery and a location on the skin proximal the right iliac artery, or
(j) a location on the skin proximal the left posterior tibial artery and a location on the skin proximal the right posterior tibial artery.

14. The method of claim 1, wherein said arteries include at least four of the following:
(a) a location on the skin proximal the left carotid artery and a location on the skin proximal the right carotid artery,
(b) a location on the skin proximal the left axillary artery and a location on the skin proximal the right axillary artery,
(c) a location on the skin proximal the left femoral artery and a location on the skin proximal the right femoral artery,
(d) a location on the skin proximal the left popliteal artery and a location on the skin proximal the right popliteal artery,
(e) a location on the skin proximal the left anterior tibial artery and a location on the skin proximal the right anterior tibial artery,
(f) a location on the skin proximal the left dorsalis pedis artery and a location on the skin proximal the right dorsalis pedis artery,
(g) a location on the skin proximal the left radial artery and a location on the skin proximal the right radial artery,
(i) a location on the skin proximal the left iliac artery and a location on the skin proximal the right iliac artery, or
(j) a location on the skin proximal the left posterior tibial artery and a location on the skin proximal the right posterior tibial artery.

15. The method of claim 1, wherein the duration of the application of optical pulses of different wavelengths in a given sequence, the duration of the application of optical pulses of different wavelengths over a plurality of sequences, the duration of the application of optical pulses in a given session, the duration of the application of optical pulses of different wavelengths in a given session, the duration of the application of optical pulses over a plurality of sessions, the duration of the application of optical pulses of different wavelengths over a plurality of sessions, the duration of the application of optical pulses over a plurality of days, the duration of the application of optical pulses of different wavelengths over a plurality of days, the number of sessions per day, the sequences or any combination thereof contribute to one or more fractal properties.

16. The method of claim 15, wherein said fractal exhibits a fractal characteristic that is within a threshold difference from a fractal characteristic of a biological signal, system, structure or process.

17. The method of claim 16, wherein said biological signal, system, structure, or process derives from or comprises water in said body.

18. The method of claim 17, further comprising using water as a universal attractor.

19. The method of claim 1, further comprising using water molecules as an extended rechargeable electrolytic biobattery.

20. The method of claim 1, wherein transmission of radiant energy through the vascular system induces coupling of photic energies supplied with multiple ligands at different time-space scales that follow a dynamic system and intervene in the restitution of physiological rhythms.
